(19) United States
(12) Patent Application Publication
(10) Pub. No.: US 2016/0346366 A1
(43) Pub. Date: Dec. 1, 2016

(54) FUSION PROTEINS COMPRISING FACTOR IX FOR PROPHYLACTIC TREATMENT OF HEMOPHILIA AND METHODS THEREOF

(71) Applicant: CSL LIMITED, Parkville, Victoria (AU)

(72) Inventors: Iris JACOBS, King of Prussia, PA (US); Debra BENSEN-KENNEDY, Phoenixville, PA (US); Christine VOIGT, Devon, PA (US)

(21) Appl. No.: 15/106,911

(22) PCT Filed: Dec. 1, 2014
(86) PCT No.: PCT/AU2014/050388

§ 371 Date: Jun. 21, 2016

Related U.S. Application Data
(60) Provisional application No. 61/919,884, filed on Dec. 23, 2013.

(51) Int. Cl. A61K 38/48; C07K 14/76; C12N 9/64; A61K 9/00

(52) U.S. Cl. CPC A61K 38/4846; A61K 9/0019; C07K 14/76; C12N 9/644; C12Y 304/21022; C07K 2319/31; C07K 2319/50

(57) ABSTRACT

The present invention relates to prophylactic dosing regimens with long-acting factor IX (FIX) in dosing intervals of 1 week or longer, including (but not limited to) 10 days or longer, such as two weeks, three weeks or even monthly.

### PRIORITY DETAILS

The present application claims priority from U.S. Provisional Patent Application No. 61/919,884 entitled “Fusion proteins comprising Factor IX for prophylactic treatment of hemophilia and methods thereof” filed on 23 Dec. 2013, the entire contents of which are hereby incorporated by reference.

### FIELD OF THE INVENTION

The present invention relates to prophylactic dosing regimens with long-acting factor IX (FIX) in dosing intervals of 1 week or longer, including (but not limited to) 10 days or longer, such as two weeks, three weeks or even monthly.

### BACKGROUND OF THE INVENTION

Hemophilia B is an X-linked recessive inherited bleeding disorder resulting from a deficiency of coagulation factor IX (FIX), a coagulation factor central to the process of blood coagulation. Signs and symptoms of hemophilia B are variable, depending on the severity of FIX deficiency and the location of bleeding. Most often, bleeding is characterized by spontaneous or trauma-induced hemorrhage into joints, muscles, and soft tissues. Recurrent bleeding in the same location may lead to permanent injury of the joint Ram but life-threatening bleeding may also occur in the central nervous system, throat, or gastrointestinal tract.

The goal of therapy for hemophilia B is to treat or prevent hemorrhage, thereby reducing disabling joint and tissue damage, and improving quality of life (QoL). Replacement therapy with FIX provides a temporary correction of the factor deficiency and reduces bleeding tendencies. Currently, plasma-derived and recombinant FIX products are used for the prophylactic and on-demand treatment of hemophilia B. However, plasma-derived products are associated with risks related to transmission of Infectious viruses such as human immunodeficiency virus, hepatitis B virus, and hepatitis C virus. Both plasma-derived and recombinant FIX products have a short half-life and, therefore, require dosing 2 to 3 times a week in order to achieve a significant reduction of bleeding episodes. In addition, repeat dosing may be required to control bleeding episodes with a relatively short bleeding-free period following administration. The need for frequent intravenous (IV) injections of either plasma-derived or recombinant FIX products carries significant burden for patients and the physicians treating their disorder. Such a regimen in younger children often (but not always), requires the insertion of a venous access device that must be kept extremely clean to avoid infectious complications and prevent the development of clots in the line. The risk and morbidity associated with such devices may prevent some very young children with hemophilia from receiving adequate care. A FIX product that has a prolonged half-life and better recovery rate may allow patients to achieve adequate homeostasis with fewer injections.

A pegylated form of FIX has been generated which shows 5 to 7 times prolonged half-life of FIX in minipigs and dogs (Østergard, H, et al., 2011. Blood. 118(8):2333-2341). Similar results were observed in patients (Negrier C et al. Blood. 2011 Sep. 8, 118(10):2695-701).

A fusion protein comprising recombinant FIX and albumin (rIX-FP) has demonstrated in rats, rabbits, and FIX-deficient mice that it has improved PK parameters (i.e., increased recovery, terminal half-life and area under the concentration-time curve [AUC]) compared with published results of a currently marketed recombinant FIX product (e.g., rFIX)) (Metznet H J, et al., 2009. Thromb Haemost. 102:634-644). Studies in humans showed that weekly prophylaxis of rIX-FP decreased the consumption of FIX compared to the previous FIX product with fewer infusions (Martinowitz et al., ISTH, Amsterdam, The Netherlands, Jun. 29-Jul. 4, 2013 abstract).

Another example of such a fusion protein comprising FIX is rFIX-Fc. In WO 2012/006624, therapeutic chimeric polypeptides comprising FIX are described which can be fused to FcRn Binding Partners such as Fc or albumin. rFIX-Fc showed an increased half-life in vivo. This document suggests administering rFIXFc at 20 IU/kg weekly, 40 IU/kg every 10 days or 120 IU/kg every two weeks for prophylactic therapy. Luk et al. (Luk A, et al., 2011. Haemophilia. 17:352-380) also describes that rFIX-Fc showed an increase in half-life, as well as in other PK parameters, compared to BeneFIX® (i.e., rFIX). The phase 3 study for rFIX-Fc was recently published which shows that when administered at a weekly interval starting at 50 IU/kg or at 100 IU/kg starting at a 10 day dosing interval, there was tow annualized bleeding rates in patients with hemophilia B (Powell J S, et al., Dec. 12, 2013. NEJM. 369:2313-23).

The present invention provides prophylactic dosing regimens with long-acting factor IX in dosing intervals of 1 week or longer, such as 10 days or longer. According to the invention described herein, even longer periods of prophylactic dosing can be achieved than previously envisioned, such as two weeks, three weeks or even monthly.

In a preferred embodiment, the present invention provides prophylactic dosing regimens for a fusion protein comprising FIX and the HLEP, wherein the Factor IX (FIX) portion is connected to the half-life enhancing polypeptide (HLEP) via a cleavable peptide linker. The HLEP (e.g., albumin) increases the half-life of FIX, but without a cleavable linker, the FIX has in general reduced or low activity. The cleavage of such linkers liberates the polypeptide from any activity compromising steric hindrance caused by the HLEP and thereby allows the generation of fusion proteins, which retain a high molar specific activity of the FIX. Such fusion proteins exhibit improved half-life and molar specific activities that are increased in comparison to their non-cleavable counterparts. Preferably, cleavage occurs by proteins involved in coagulation. This allows the non-activated FIX to have an increased half-life until a bleeding event occurs, and simultaneous activation of FIX and cleavage from HLEP (e.g., albumin). The activated FIX, which is liberated from its fusion to the HLEP, has both high activity and is rapidly cleared from the blood due to the loss of HLEP (e.g., albumin). This rapid clearance is desirable since extended time of the activated FIX might lead to thrombotic complications.

The prior art describes a fusion protein comprising FIX and albumin with a non-cleavable linker. For example, WO 01/179271 describes fusion polypeptides of a multitude of different therapeutic polypeptides which, when fused to human serum albumin, are predicted to have an increased functional half-life in vivo and extended shelf-life. Among the list of therapeutic polypeptides mentioned as Examples is Factor IX. Also described are fusions of FIX in which there is a peptide linker between albumin and FIX, but the linker is not specified to be cleavable. Sheffield et al. (Sheffield W. P. et al. (2004), Br. J. Haematol. 126: 565-573) expressed a murine Factor IX albumin fusion protein composed of murine FIX, a linker of 8 amino acids (GPG₄TM), murine albumin and a peptide tag of 22 amino acids, and also a human Factor IX albumin fusion protein composed of human Factor IX, a linker of 7 amino acids (G₆V) and human albumin. Sheffield does not use or suggest using a cleavable linker between FIX and albumin. These fusion proteins allow FIX to be activated but it has low activity due to the presence of albumin.

Once a coagulation factor is activated during coagulation either by proteolytic cleavage of the zymogen (like FIX) or by contact of an already proteolytically “pre”-activated factor to a second polypeptide (like FVIIa binding to Tissue Factor), it is no longer desirable to maintain the long half-life of the now activated coagulation factor, as this might lead to thrombotic complications and should be even more relevant if the activated factor would have an increased half-life. It is therefore one objective of the present invention to provide long-lived FIX suitable for prophylactic therapy with treatment periods of 1 week or longer and even as long as two weeks, three weeks and even monthly.

Fusions of the coagulation factors to half-life enhancing polypeptides as described in the prior art suffer in general from a reduced molar specific activity of the fused coagulation factor. Another aspect of the present invention to provide coagulation factors with enhanced half-life, which show increased molar specific activity compared to the corresponding therapeutic fusion protein without a cleavable linker.

US2008/0260755 describes fusion proteins comprising a coagulation factor, such as FIX, and a half-life enhancing polypeptide, connected by a cleavable peptide linker that may be cleaved by a protease involved in coagulation. These fusion proteins have increased half-lives and molar specific activity. This application is herein incorporated by reference in its entirety.

An example of the fusion protein of the invention is a fusion protein comprising FIX and albumin where the cleavable linker is cleavable by a protease involved in coagulation. Proteolytic cleavage in a coagulation-related mode, in the sense of the invention, is any proteolytic cleavage that occurs as a consequence of the activation of at least one coagulation factor or coagulation cofactor. The coagulation factor is activated almost in parallel to the proteolytic cleavage of the linker peptide. Activation may occur, for example by proteolytic cleavage of the coagulation factor or by binding to a cofactor. This results in activation of FIX with a long half-life and high activity upon activation (see FIG. 1). The albumin increases the half-life of FIX in the blood until a bleeding event occurs, the bleeding event simultaneously activates FIX and cleaves it from albumin so the cleaved FIX has both high activity and is rapidly cleared from the blood due to the loss of albumin.

rIX-FP (rFIX-albumin fusion protein) having the sequence set forth in SEQ ID NO: 1 (see FIG. 2), showed improved PK parameters. Specifically, it has prolonged circulation in plasma as shown by the 5.3-fold longer half-life (t_{1/2}), the 7-fold reduced CL, and the 7-fold greater AUC compared to rFIX (e.g., Benefix®) (Santagostino E, et al. Blood. 2012 Sep. 20; 120(12):2405-11). This surprisingly allows for prophylactic treatment of hemophilia with dosing intervals that are significantly longer than suggested by the prior art for rFIX.

The technical advantage of the present invention is that the fusion protein (e.g., rFIX-FP) has both a longer half-life and over 30% higher incremental recovery than other known FIX products. The higher incremental recovery of the fusion protein means that a lower dose (i.e., less protein) is administered to achieve a necessary FIX activity level, based on the standard dosing formula:

$$\begin{matrix}Number \\ of factor \\ IX IU \\ required \\ ( IU )\end{matrix} = \begin{matrix}Body \\ weight \\ ( kg )\end{matrix} \times \begin{matrix}Desired \\ factor IX \\ increase \\ ( \% or \\ IU \text{/} dL )\end{matrix} \times \begin{matrix}Reciprocal of \\ observed \\ recovery \\ ( IU \text{/} kg per \\ IU \text{/} dL )\end{matrix}$$

Therefore, the fusion protein (e.g., rIX-FP) is less likely to be immunogenic since fewer host cell proteins are administered, in addition to a lower risk of local reactions at the site of injection. Longer half-life, lower clearance, larger AUC (enhanced exposure) and higher incremental recovery benefit patients since less product (IU/kg per dose and IU/kg per week) is used and less frequent administrations are required to achieve the same FIX peak and trough activity levels in a patient.

### SUMMARY OF THE INVENTION

The invention relates to a fusion protein comprising
a) a Factor IX (FIX) portion, and
b) a half-life enhancing polypeptide (HLEP)

for use in a method of preventing bleeding in a subject, wherein the fusion protein is to be administered to the subject at a dose of about 25-75 IU/kg for a dosing interval of about once every week. In a preferred embodiment, the dose is about 35-75 IU/kg. In another more preferred embodiment, the dose is about 35-55 IU/kg. In still another more preferred embodiment, the dose is about 35-50 IU/kg. In still another more preferred embodiment, the dose is about 25-50 IU/kg. In another preferred embodiment, the dose is 30-40 IU/kg. In a preferred embodiment, the dose is about 50 IU/kg. In another preferred embodiment, the dose is about 45 IU/kg. In still another highly preferred embodiment, the dose is about 40 IU/kg. In another highly preferred embodiment, the weekly dose is about 35 IU/kg. In still another highly preferred embodiment, the weekly dose is about 25 IU/kg. In the most preferred embodiment, the weekly dose is about 25-40 IU/kg.

In any one of these embodiments, the dosing interval may be about once every 6 to 8 days, preferably about once every 7 days. In any one of these embodiments, the HLEP may be an HLEP that is not PC. In any one of these embodiments, the HLEP is preferably albumin. In preferred embodiments, the plasma level of the FIX is maintained at a trough of at least about 1%, preferably at least about 2%, preferably at least about 3%, preferably at least about 4%, or preferably at least about 5% above baseline for the entire dosing interval, and more preferably between 4% and 15%, or even more preferably between 5 and 15% above baseline for the entire dosing interval.

The invention also relates to a fusion protein comprising
a) a Factor IX (FIX) portion, and
b) a half-life enhancing polypeptide (HLEP)

for use in a method of preventing bleeding in a subject, wherein the fusion protein is to be administered to the subject at a dose of about 50-95 IU/kg for a dosing interval of about once every 8 to 11 days. In a preferred embodiment, the dose is 50-75 IU/kg. In this preferred embodiment, the dose can be about 75 IU/kg. In this preferred embodiment, the dose can be about 50 IU/kg. Preferably, the dosing interval is about once every 10 days. In another embodiment, the dose is administered 3 times per month.

The invention further relates to a fusion protein comprising
a) a Factor IX (FIX) portion, and
b) a half-life enhancing polypeptide (HLEP)

for use in a method of preventing bleeding in a subject, wherein the fusion protein is to be administered to the subject at a dose of about 50-95 IU/kg for a dosing interval of about once every two weeks. In a preferred embodiment, the dose is 65-85 IU/kg. In another preferred embodiment, the dose is about 60-80 IU/kg. In still another preferred embodiment, the dose is about 70-80 IU/kg. In the most preferred embodiment, the dose is about 50-75 IU/kg. In this most preferred embodiment, the dose can be about 75 IU/kg or the dose can be about 50 IU/kg. In any one of these embodiments, the dosing interval may be about once every 12 to 16 days, preferably about once every 13 to 15 days, more preferably about once every 14 days.

The invention further relates to a fusion protein comprising.
a) a Factor IX (FIX) portion, and
b) a half-life enhancing polypeptide (HLEP)

for use in preventing bleeding in a subject, wherein the fusion protein is to be administered to the subject at a dose of at least about 90-250 IU/kg for a dosing interval of about once every 3 weeks or longer. In a preferred embodiment, the dose is about 90-150 IU/kg. In another preferred embodiment, the dose is about 95-110 IU/kg. In another preferred embodiment, the dose is about 95-105 IU/kg. In a more preferred embodiment, the dose is about 100 IU/kg. In any of these embodiments, the dosing interval may be about once every 19 to 23 days, preferably about once every 20 to 22 days, more preferably about once every 21 days. Alternatively, the dosing interval may be about once every month. In a preferred embodiment, the dose is about 140-200 IU/kg and the dosing interval is about once every month. In a more referred embodiment, the dose is about 140-160 IU/kg and the dosing interval is about once every month. In a highly preferred embodiment, the dose is about 150 IU/kg and the dosing interval is about once every month. In any of these embodiments, the dosing interval may be about once every 28 days.

In any one of the embodiments of the invention, the half-life enhancing polypeptide (HLEP) may be albumin (FP) or an immunoglobulin without an antigen binding domain (e.g., Fc). In a preferred embodiment, the half-life enhancing polypeptide. (HLEP) is albumin (FP). In one particular embodiment, the HLEP is not F.

In a preferred embodiment, the Factor IX (FIX) portion of the fusion protein is connected to the half-life enhancing polypeptide (HLEP) via a peptide linker. In another highly preferred embodiment, the peptide linker is cleavable. In an even more preferred embodiment, the peptide linker is cleavable by proteases involved in coagulation or activated by coagulation enzymes. Proteases involved in coagulation are activated once the coagulation cascade is activated which ultimately results in the generation of fibrin from fibrinogen.

In the most preferred embodiments of the invention, the linker is cleavable by the protease that activates the coagulation factor, thereby ensuring that the cleavage of the linker is linked to the activation of the coagulation factor at a site at which coagulation occurs. Other preferred fusion proteins, according to the invention, are those wherein the linker is cleavable by the coagulation factor which is part of the fusion protein once it is activated, thus also ensuring that cleavage of the fusion protein is connected with a coagulatory event. Other preferred fusion proteins according to the invention are those, wherein the linker is cleavable by a protease, which itself is activated directly or indirectly by die activity of the coagulation factor which is part of the fusion protein, thus also ensuring that cleavage of the fusion protein is connected with a coagulatory event.

In a preferred embodiment, the linker is cleavable by FIXa and/or by FVIIa/Tissue Factor (TF).

In a particularly preferred embodiment, the linker comprises as sequence selected from SEQ ID NO: 2 and SEQ ID NO: 3:

```
SEQ ID NO: 2
Pro Val Ser Gln Thr Ser Lys Leu Thr Arg Ala
1 5 10
Glu Thr Val Phe Pro Asp Val
                        15
SEQ ID NO: 3
Pro Ser Val Ser Gln Thr Ser Lys Leu Thr Arg
1 5 10
Ala Glu Thr Val Phe Pro Asp Val
                        15
```

In an alternative embodiment, the linker is 90% identical to one of SEQ ID NO: 2 and SEQ ID NO: 3. In another embodiment, it is 80% identical to one of SEQ ID NO: 2 and SEQ ID NO: 3. In still another embodiment, it is 70% identical to one of SEQ ID NO: 2 and SEQ ID NO: 3. In still another embodiment, it is 60% identical to one of SEQ ID NO: 2 and SEQ ID NO: 3. In a further embodiment, it is 50% identical to one of SEQ ID NO: 2 and SEQ ID NO: 3.

Preferably, the fusion protein of the invention has the sequence as set forth in SEQ ID NO: 1 (see FIG. 2). Alternatively, the sequence of the fusion protein has at least 70% identity to the sequence set forth in SEQ ID NO: 1. The sequence of the fusion protein may have at least 75% identity to the sequence set forth in SEQ ID NO: 1. The sequence of the fusion protein may have at least 80% percent identity to the sequence set forth in SEQ ID NO: 1. The sequence of the fusion protein may have at least 85% percent identity to the sequence set forth in SEQ ID NO: 1. The sequence of the fusion protein may have at least 90% percent identity to the sequence set forth in SEQ ID NO: 1. The sequence of the fusion protein may have at least 95% percent identity to the sequence set forth in SEQ ID NO: 1. The sequence of the fusion protein may have at least 98% percent identity to the sequence set forth in SEQ ID NO: 1. The sequence of the fusion protein may have at least 99% percent Identity to the sequence set forth in SEQ ID NO: 1.

For any of the embodiments of the invention, the plasma level of the FIX is maintained at a trough of at least about 1% above baseline for the entire dosing interval. Preferably, the plasma level of the FIX is maintained at a trough of at least about 2-5% above baseline for the entire dosing interval, and more preferably at least about 2, 3, 4, or 5% above baseline for the entire dosing interval. In other more preferred embodiments the plasma level of the FIX is maintained between 4 and 15% or between 5 and 15% above baseline for the entire dosing interval.

For the purposes of the invention, the preferred subject to be administered the fusion protein is human. Particularly preferred is a human that suffers from hemophilia B.

The fusion proteins of the invention may be for use as in a treatment involving a prophylactic dosing regimen. In a particularly preferred embodiment, the dose is to be administered intravenously.

Also encompassed by the present invention is a method of administering Factor IX (FIX) to a subject in need thereof, comprising administering to the subject a dose of about 25-75 IU/kg of a fusion protein comprising
a) a Factor IX (FIX) portion, and
b) a half-life enhancing polypeptide (HLEP)

at about a once weekly or longer dosing interval. In a preferred embodiment, the dose is about 35-75 IU/kg. In another more preferred embodiment, the dose is about 35-55 IU/kg. In another more preferred embodiment, the dose is about 35-50 IU/kg. In another more preferred embodiment, the dose is about 25-50 IU/kg. In still another preferred embodiment, the dose is 30-40 IU/kg. In a preferred embodiment, the dose is about 50 IU/kg. In another preferred embodiment, the dose is about 45 IU/kg. In another highly preferred embodiment, the dose is about 40 IU/kg. In another highly preferred embodiment, the dose is about 35 IU/kg. In another highly preferred embodiment, the dose is about 25 IU/kg. In the most preferred embodiment, the weekly dose is about 25-40 IU/kg. In any one of these embodiments, the dosing interval may be about once every 6 to 8 days, preferably about once every 7 days. In any one of these embodiments, the HLEP may be an HLEP that is not Fc. In any one of these embodiments, the HLEP is preferably albumin.

In preferred embodiments, the plasma level of the FIX is maintained at a trough of at least about 1%, preferably at least about 2-5% above baseline for the entire dosing interval, and more preferably at least about 2, 3, 4, or 5% above baseline for the entire dosing interval. In other more preferred embodiments the plasma level of the FIX is maintained between 4 and 15% and more preferably between 5 and 15% above baseline for the entire dosing interval.

The invention also relates to method of administering Factor IX (FIX) to a subject in need thereof, comprising administering to the subject a dose of about 50-95 IU/kg of a fusion protein comprising
a) a Factor IX (FIX) portion, and
b) a half-life enhancing polypeptide (HLEP)

at a dosing interval of about once every 8 to 11 days. In a preferred embodiment, the dose is about 50-75 IU/kg. In this more preferred embodiment, the dose is about 75 IU/kg or alternatively, in this preferred embodiment the dose may be 50 IU/kg. Preferably, the dosing interval is about once every 10 days. In another embodiment, the dose is administered 3 times per month.

Another embodiment of the invention relates to a method of administering Factor IX (FIX) to a subject in need thereof, comprising administering to the subject a dose of about 50-95 IU/kg of a fusion protein comprising
a) a Factor IX (FIX) portion, and
b) a half-life enhancing polypeptide (HLEP)

at about a once every two weeks or longer dosing interval. In a preferred embodiment, the dose is 50-75 IU/kg. In another preferred embodiment, the dose is 65-85 IU/kg. In yet another preferred embodiment, the dose is 60-80 IU/kg. In still another preferred embodiment, the dose is about 70-80 IU/kg. In the most preferred embodiment, the dose is about 50-75 I/kg. In this most preferred embodiment, the dose may be about 50 IU/kg, or in the most preferred embodiment, the dose is about 75 IU/kg. In any of these embodiments, the dosing interval may be about once every 12 to 16 days, preferably about once every 13 to 15 days, more preferably, about once every 14 days.

Also encompassed by the invention is a method of administering Factor IX (FIX) to a subject in need thereof, comprising administering to the subject a dose of 90-250 IU/kg of a fusion protein comprising
a) a Factor IX (FIX) portion, and
b) a half-life enhancing polypeptide (HLEP)

at about a once every 3 week or longer dosing interval. In a preferred embodiment, the dose is about 90-150 IU/kg. In another preferred embodiment, the dose is about 95-110 IU/kg. In another preferred embodiment, the dose is about 95-105 IU/kg. In the most preferred embodiment, the dose is about 100 IU/kg. In any of these embodiments, the dosing interval may be about once every about 19 to 23 days, preferably about once every 20 to 22 days, more preferably about once every 21 days. Alternatively, the dosing interval may be about once every month. In a preferred embodiment, the dose is about 140-200 IU/kg and the dosing interval is about once every month. In a more preferred embodiment, the dose is about 140-160 IU/kg and the dosing interval is about once every month. In a highly preferred embodiment, the dose is about 150 IU/kg and the dosing interval is about once every month. In any of these embodiments, the dosing interval may be about once every 28 days.

In any of the above embodiments, the plasma level of the FIX is maintained a trough of at least about 1%, preferably at least about 2-5%, more preferably 2-4% above baseline for the entire dosing interval, and even more preferably at least about 2, 3, 4%, or $% above baseline for the entire dosing interval. In other preferred embodiments, the plasma level of the FIX may also be maintained between 5 and 15% above baseline for the entire dosing interval. In the most preferred embodiment, the plasma level of the FIX is maintained a trough of at least 4% above baseline for the entire dosing interval for up to 21 days. For example, using a 100 IU/kg prophylaxis dosing regimen in hemophilia patients, a FIX activity was shown above baseline at 21 days post-injection with a preferred fusion protein of the invention (rIX-FP) (see Table 2, FIGS. 8 and 9).

For any of the methods of the invention, the half-life enhancing polypeptide (HLEP) may be albumin (FP) or an immunoglobulin without an antigen binding domain (e.g., Fc). In a preferred embodiment, the half-life enhancing polypeptide (HLEP) is albumin (FP). In one particular embodiment, the HLEP is not FC.

In a highly preferred embodiment, the Factor IX (FIX) portion of the fusion protein is connected to the half-life enhancing polypeptide (HLEP) via a peptide linker. In another highly preferred embodiment, the peptide linker is cleavable. In an even more preferred embodiment, the peptide linker is cleavable by proteases involved in coagulation or activated by coagulation enzymes. Proteases involved in coagulation are activated once the coagulation cascade is activated which ultimately results in the generation of fibrin from fibrinogen.

In the most preferred embodiments of the invention, the linker is cleavable by the protease that activates the coagulation factor, thereby ensuring that the cleavage of the linker is linked to the activation of the coagulation factor at a site at which coagulation occurs. Other preferred fusion proteins, according to the invention, are those wherein the linker is cleavable by the coagulation factor which is part of the fusion protein once it is activated, thus also ensuring that cleavage of the fusion protein is connected with a coagulatory event. Other preferred fusion proteins according to the invention are those, wherein the linker is cleavable by a protease, which itself is activated directly or indirectly by the activity of the coagulation factor which is part of the fusion protein, thus also ensuring that cleavage of the fusion protein is connected with a coagulatory event.

In a preferred embodiment, the linker is cleavable by FIXa and/or by FVIIa/Tissue Factor (TF).

In particularly preferred methods of the invention, the linker comprises a sequence selected from SEQ ID NO: 2 and 3:

```
SEQ ID NO: 2
Pro Val Ser Gln Thr Ser Lys Leu Thr Arg Ala
1 5 10
Glu Thr Val Phe Pro Asp Val
                        15
SEQ ID NO: 3
Pro Ser Val Ser Gln Thr Ser Lys Leu Thr Arg
1 5 10
Ala Glu Thr Val Phe Pro Asp Val
                        15
```

In an alternative embodiment, the linker is 90% identical to one of SEQ ID NO: 2 and SEQ ID NO: 3. In another embodiment, it is 80% identical to one of SEQ ID NO: 2 and SEQ ID NO: 3. In still another embodiment, it is 70% identical to one of SEQ ID NO: 2 and SEQ ID NO: 3. In still another embodiment. It is 60% identical to one of SEQ ID NO: 2 and SEQ ID NO: 3. In a further embodiment, it is 50% identical to one of SEQ ID NO: 2 and SEQ ID NO: 3.

Preferably, the methods of the invention administer a fusion protein, which has the sequence as set forth in SEQ ID NO: 1 (see FIG. 2). Alternatively, the sequence of the fusion protein has at least 70% identity to the sequence set forth in SEQ ID NO: 1. The sequence of the fusion protein may have at least 75% identity to the sequence set forth in SEQ ID NO: 1. The sequence of the fusion protein may have at least 80% percent identity to the sequence set forth in SEQ ID NO: 1. The sequence of the fusion protein may have at least 85% percent identity to the sequence set forth in SEQ ID NO: 1. The sequence of the fusion protein may have at least 90% percent identity to the sequence set forth in SEQ ID NO: 1. The sequence of the fusion protein may have at least 95% percent identity to the sequence set forth in SEQ ID NO: 1. The sequence of the fusion protein may have at least 98% percent identity to the sequence set forth in SEQ ID NO: 1. The sequence of the fusion protein may have at least 99% percent identity to the sequence set forth in SEQ ID NO: 1.

For any of the methods of the invention, the plasma level of the FIX is maintained a trough of at least about 1% above baseline for the entire dosing interval Preferably, the plasma level of the FIX is maintained at a trough of at least about 1% above baseline for the entire dosing interval.

For the methods of the invention, the preferred subject is human. Particularly preferred is a human that suffers from hemophilia B.

The methods of the invention may be for treatment involving a prophylactic dosing regimen. In a particularly preferred method of the invention, the dose is administered intravenously.

For any of the embodiments of the invention, the fusion protein is preferably provided for administration at a concentration of about 100 to 400 IU/ml, preferably about 100, 200 or 400 IU/ml. The fusion protein may also be provided for administration at concentrations of 600 IU/mL or 1200 IU/ml.

### DETAILED DESCRIPTION OF THE INVENTION

“Prophylactic treatment”, as used herein, means administering a Factor IX fusion protein in multiple doses to a subject over a course of time to increase the level of Factor IX activity in a subject's plasma. Preferably, the increased level is sufficient to decrease the incidence of spontaneous bleeding or to prevent bleeding in the event of an unforeseen injury. Prophylactic treatment decreases or prevents bleeding episodes, for example, those described under on-demand treatment. Prophylactic treatment may be fixed or may be individualized, as discussed under “dosing interval”, e.g., to compensate for inter-patient variability.

“Dosing interval”, as used herein, means the amount of time that elapses between multiple doses being administered to a subject. The dosing interval in the methods of the invention using a chimeric FIX-HLEP, e.g., FIX-FP, may be at least about one and one-half to eight times longer than the dosing interval required for an equivalent amount (in IU/kg) of said Factor IX without the HLEP, e.g., albumin (i.e., a polypeptide consisting of said FIX). The dosing interval when administering, e.g., a Factor IX-HLEP fusion protein of the invention may be at least about one and one-half-times to eight times longer than the dosing interval required for an equivalent amount of said Factor IX without the HLEP, e.g., albumin. The dosing interval may be at least about one and one-half to eight times longer than the dosing interval required for an equivalent amount of said Factor IX without, e.g., albumin (or a polypeptide consisting of said Factor IX).

“Median dose”, as used herein, means half of the study subjects used higher than that dose and half of the study subjects used lower than that dose. “Mean dose” means an average dose (is computed by adding up all the doses and dividing by the total number of the doses). For a given dose, “about” means the dose indicated plus or minus 1, 2, 5, 10, 15 or 20% of that indicated dose. For a dosing interval of about once every 6 to 8 days or about once every 7 days, “about” means plus or minus 12 hours. For a dosing interval of about once every 8 to 11 days, or about once every 10 days, “about” means plus or minus 18 hours. For a dosing interval of about three times per month, about once every two weeks, about once every 13 to 15 days, about once every 14 days, or about twice per month, “about” means plus or minus 1 day. For a dosing interval of about once every 3 weeks or longer, about once every 20 to 22 days, about once every 21 days, or about once every month, “about” means plus or minus 2 days.

“Maintaining the plasma level of FIX at a trough of at least about” a certain percentage means, that the FIX biological activity in plasma will not fall below said percentage level during a certain dosing regimen of a patient in need of FIX, wherein 100% of said FIX biological activity correspond to 1 U/ml which is the FIX activity concentration in normal human plasma, and wherein the FIX biological activity preferably is determined using a validated one-stage clotting method as described in the Examples.

The “trough” is the lowest level of said FIX biological activity throughout the dosing regimen during the treatment of a patient in need of FIX. Due to patient inter-variability, the trough level generally refers to median values, which means that half of the study subjects had a higher trough level and half of the study subjects bad a lower trough level see e.g. FIGS. 7 and 12. Though using the median value is more common, the trough level from the PK data could also be calculated as a mean value, which have been determined by adding up the values for all patients and dividing by the number of patients, see e.g. Table 8.

“Baseline” means the FIX activity level in a given patent preferentially expressed in IU/dL or in % of the FIX activity in a healthy person which is defined to be 100 IU/dL or 100%. In severe hemophilia B the baseline level of a given patient is very low to zero or almost zero, whereas in mild hemophilia the patient's baseline may be higher such as above 1%, above 2% or above 3% or above 4% or above 5% of the FIX activity concentration in a healthy person. When the fusion protein comprising i) a Factor IX (FIX) portion, and ii) a half-life enhancing polypeptide (HLEP) is administered according to the present invention first the FIX activity concentration sharply increases and is the slowly cleared i.e., it is returning to the individual baseline level. Especially in severe hemophilia B cam has to be taken that the FIX activity concentration does not fall below a minimal level in order to prevent bleeding. This minimal level is called the trough level. In a severe hemophilia B patient when the baseline is practically zero a trough level of 1% above baseline means a FIX activity concentration of about 1% of the FIX activity concentration in a healthy person. It a mild hemophilia B patient having a baseline level of 3% FIX activity of the FIX activity concentration in a healthy person a trough level of 1% above baseline means a FIX activity concentration of about 4% of the FIX activity concentration in a healthy person.

In the invention, the dosing interval may be about once every week, about once every 6 to 8 days, about once every 7 days, about once every 8 to 11 days, about once every 10 days, about three times per month, about once every two weeks, about once every 13 to 15 days, about once every 14 days, about twice per month, about once every 3 weeks or longer, about once every 20 to 22 days, about once every 21 days, or about once every month. In particular dosing intervals of 1 week, 2 weeks, 3 weeks, and even one month, are contemplated. The most preferred dosing intervals are one week (7 days), two weeks (14 days) or three weeks (21 days).

The dosing interval may, alternatively, be an individualized interval that is determined for each subject based on pharmacokinetic data or other information about that subject. The individualized dose/dosing interval combination may be the same as those for fixed interval regimens in the preceding paragraphs, or may differ. The regimen may initially be at a fixed dosing interval, and then it may change to an individualized dosing interval. The regimen may initially be at a fixed dose (IU/kg) and dosing interval, and then it may change to an individualized dosing interval with the fixed dose. The regimen may also initially be at a fixed dosing interval and dose (IU/kg), and then it may change to an individualized dose with the same fixed dosing interval.

The therapeutic doses that may be used in the methods of the invention are about 25-75 IU/kg, about 35-75 IU/kg, about 30-50 IU/kg, about 35-55 IU/kg, about 35-50 IU/kg, about 25-50 IU/kg, about 50 IU/kg, about 45 IU/kg, about 35 IU/kg for weekly dosing. A median/mean dose of about 40 IU/kg has been observed in the on-going study. A dose of 35 IU/kg is contemplated for the weekly dosing regimen. For a dosing schedule of once of every two weeks doses of 50-75 IU/kg, about 50 IU/kg, about 75 IU/kg, about 60-90 IU/kg, about 65-85 IU/kg, about 70-80 IU/kg, about 75 IU/kg are envisioned. A fixed dose of 75 IU/kg has been observed with excellent efficacy in the on-going study. Notably, the prior art has not disclosed that a dose as low as 50-75 IU/kg can be used at a dosing interval of two weeks.

For a three week dosing interval, the invention contemplates doses of, about 90-250 IU/kg, about 90-150 IU/kg, about 95-110 IU/kg, about 95-105 IU/kg, and about 1.00 IU/kg. For a monthly dosing interval, the invention contemplates a dose of 140-250 IU/kg. The prior art has not disclosed that a three week or monthly dosing interval can be achieved.

Accordingly, preferred therapeutic doses are about 35-75 IU/kg, about 35-55 IU/kg, about 35-50 IU/kg, about 25-50 IU/kg, about 30-60 IU/kg, about 30-50 IU/kg, about 30-40 IU/kg about 50 IU/kg, about 45 IU/kg, about 35 IU/kg, about 75 IU/kg, about 70-80 IU/kg, about 75 IU/kg, about 9-110 IU/kg, about 95-105 IU/kg, about 100 IU/kg, and about 140-250 IU/kg. A prophylactic dose(s) should not exceed 250 IU/kg monthly.

Preferred doses and dosing intervals are as follows: about 25-75 IU/kg about once every week, about 35-75 IU/kg about once every week, about 35-55 IU/kg about once every week, about 35-50 IU/kg about once every week, about 25-50 IU/kg about once every week, about 50 IU/kg about once every week, about 45 IU/kg about once every week, and about 35 IU/kg about once every week. “About once every week” includes about once every 6 to 8 days and about once every 7 days.

Other preferred doses and dosing intervals are: about 50-75 IU/kg about once every 8 to 11 days, about 50 IU/kg about once every 8 to 11 days, and about 75 IU/kg about once every 8 to 11 days. “About once every 8 to 11 days” includes about every once every 10 days.

Still other preferred doses and dosing intervals are: about 50-90 IU/kg about once every two weeks, about 50-75 IU/kg about once every two weeks, about 65-85 IU/kg about once every two weeks, about 60-80 IU/kg, about 70-80 IU/kg about once every two weeks, about 50 IU/kg about once every two weeks, and most preferably about 75 IU/kg about once every two weeks. “About once every two weeks” includes about once every 12 to 16 days or about once every 13 to 15 days, preferably about once every 14 days.

Still other preferred doses and dosing intervals are: about 90-250 IU/kg about once every 3 weeks or longer, about 90-150 IU/kg, about 80-120 IU/kg, about 95-110 IU/kg about once every 3 weeks or longer, about 95-105 IU/kg about once every 3 weeks or longer, and about 100 IU/kg about once every 3 weeks or longer. “About once every 3 weeks or longer” includes about once every 19 to 23 days about once every 20 to 22 days, preferably about once every 21 days, and about once every month.

In the invention, it is also contemplated that once-monthly dose can be administered. Thus, another preferred dose and dosing interval is about 140-250 IU/kg about once every month.

A dose of 25-40 IU/kg will be recommended for the one week (7 day) dosing regimen and is most preferred for the one week regimen. A dose of 50-75 IU/kg will be recommended for the two weeks (14 day) dosing regimen and is most preferred for the two-week regimen. A dose of 100 IU/kg is most preferred for the three-week (21 day) dosing regimen.

Surprisingly, the preferred fusion protein of the invention (rIX-FP) has so far been the only product dosed 50 IU/kg once every 7 days or longer that maintains a trough of 3% or higher in all patients, including children. Moreover, a 21-day prophylaxis regimen showed a FIX activity of at least 4% at day 21-post 100 IU/kg rIX-FP injection. The results are summarized in the Table 8. It has previously not been possible to achieve such high FIX levels for such a prolonged period of time with prior art FIX products.

Encompassed in this invention is an embodiment, where the median plasma level of FIX activity maintains a trough of at least about 5% above baseline for the entire dosing interval of 7 days after administration of a 25 IU/kg rIX-FP dose. In another embodiments the median plasma level of FIX activity maintains a trough of at least about 5% above baseline for the dosing interval of 10 days after application of a 50 IU/kg rIX-FP dose. In still another embodiment, the median plasma level of FIX activity maintains a trough of at least about 5% above baseline for the dosing interval of 14 days after application of a 75 IU/kg rIX-FP dose.

Also encompassed in this invention is an embodiment, where a 25 IU/kg rIX-FP dose is applied every 7 days, a median plasma level of FIX activity of at least about 7% above baseline is maintained for the entire dosing interval of one week. In another embodiment, where a 50 IU/kg rIX-FP dose is applied every 10 days, the median plasma level of FIX activity maintains a trough of at least about 9% above baseline for the entire dosing interval. In another embodiment, where a 50 IU/kg rIX-FP dose is applied every 14 days, the median plasma level of FIX activity maintains a trough of at least about 4% above baseline for the entire dosing interval. In another embodiment, where a 75 IU/kg rIX-FP dose is applied every 14 days, the median plasma level of FIX activity maintains trough of at least about 7% above baseline for the entire dosing interval (see e.g. Table 9b). In another embodiment, where a 50 IU/kg rIX-FP dose is applied every 14 days, the median plasma level of FIX activity maintains a trough of at least about 4% above baseline for the entire dosing interval. In another embodiment, where a 75 IU/kg rIX-FP dose is applied every 14 days, the median plasma level of FIX activity maintains trough of at least about 7% above baseline for the entire dosing interval. In another embodiment, where a 75 IU/kg rIX-FP dose is applied every 21 days, the median plasma level of FIX activity maintains trough of at least about 2% above baseline for the entire dosing interval (see e.g. Table 9b). In another embodiment, where a 100 IU/kg rIX-FP dose is applied every 21 days, the median plasma level of FIX activity maintains trough of at least about 4% above baseline for the entire dosing interval.

Alternatively, in these embodiments, the plasma level of FIX activity could be calculated as a mean (see e.g. Table 8).

In particular, the present invention provides prophylactic dosing regimens for a fusion protein comprising FIX and the HLEP albumin, wherein the Factor IX (FIX) portion is connected to the half-life enhancing polypeptide (HLEP) via a cleavable peptide linker. The dosing interval can be 1 week or longer, such as 10 days or longer but even longer periods of prophylactic dosing can be achieved than previously envisioned, such as two weeks, three weeks or even monthly.

rIX-FP fusion proteins with no linker or an non-cleavable linker allow an increased half-life of FIX due to the presence of albumin. However, since activated FIX is still fused to albumin. It has the disadvantage of having a reduced activity. In addition, because the activated FIX is still fused to albumin, it continues to have a long half-life even after the bleeding event is resolved, rIX-FP fusion proteins with a linker which allows cleavage before activation of FIX, show an increased half-life of FIX due to albumin but since cleavage occurs before the bleeding event, the half-life of activated FIX is reduced before activation. If on the other hand, the cleavage occurs after activation of FIX, the albumin increases the half-life of FIX but the activated FIX is still fused to albumin, so it has low activity.

The invention preferably relates to a fusion protein comprising FIX and albumin where the cleavable linker is cleavable by a protease involved in coagulation. Proteolytic cleavage in a coagulation-related mode, in the sense of the invention, is any proteolytic cleavage that occurs as a consequence of the activation of at least one coagulation factor or coagulation cofactor. The coagulation factor is activated almost in parallel to the proteolytic cleavage of the linker peptide (see FIG. 1). Activation may occur, for example by proteolytic cleavage of the coagulation factor or by binding to a cofactor. The albumin increases the half-life of FIX in the blood until a bleeding event occurs, the bleeding event simultaneously activates FIX and cleaves it from albumin. The cleavage liberates the polypeptide from any activity-compromising steric hindrance caused by the HLEP and thereby allows the generation of fusion proteins, which retain a high molar specific activity of the FIX. The cleaved FIX is then rapidly cleared from the blood due to the loss of albumin. Such fusion proteins exhibit improved half-life and molar specific activities that are increased in comparison to their non-cleavable counterparts. Thus, less rIX-FP is needed to provide a therapeutic effect compared to non-cleavable fusion proteins comprising rIX.

Preferred fusion proteins according to the invention are those that have a molar specific activity, in particular a molar specific coagulation-related activity of the therapeutic fusion protein that is increased at least 25% compared to that of the therapeutic fusion protein linked by a non-cleavable linker having the amino acid sequence GGGGGGV (SEQ ID NO: 4) in at least one coagulation-related assay. More preferred are fusion proteins in which the molar specific activity is increased by at least 50%, even more preferred those in which the molar specific activity is increased by at least 100%, in at least one of the different coagulation-related assays available.

In a further embodiment, the linker peptide comprises cleavage sites for more than one protease. This can be achieved either by a linker peptide that can be cleaved at the same position by different proteases or by a linker peptide that provides two or more different cleavage sites. There may be advantageous circumstances where the therapeutic fusion protein must be activated by proteolytic cleavage to achieve enzymatic activity and where different proteases may contribute to this activation step. Activation of FIX can either be achieved by FXIa or by FVIIa/Tissue Factor (TF). In a preferred embodiment, the linker is cleavable by FIXa and/or by FVIIa/Tissue Factor (TF).

rIX-PP (rFIX-albumin fusion protein) having the sequence set forth in SEQ ID NO: 1, has prolonged circulation in plasma as shown by the 5.3-fold longer half-life (t_{1/2}), the 7-fold reduced CL, and the 7-fold greater AUC compared to rFIX (e.g., Benefix®).

Moreover, when comparing pharmacokinetic parameters of rIX-FP to the corresponding pharmacokinetic data of FIX-Fc in the Alprolix™ FDA prescribing information, rIX-FP has an about 3.8-fold higher AUC0-inf, an about 3.7-fold reduced CL/BW, an about 26% higher incremental recovery, an about 3.2-fold lower volume of distribution, and an increased mean residence time of the drug by about 19%, when compared to rIX-Fc (ALPROLIX™).

Prophylaxis is the treatment by intravenous injection of factor concentrate in order to prevent anticipated bleeding. Prophylaxis was conceived from the observation that moderate hemophilia patients with clotting factor level >1 IU/dl (>19%) seldom experience spontaneous bleeding and have much better preservation of joint function. Therefore, prophylaxis with FIX activity maintained above 1% to prevent bleeding and joint destruction should be the goal of therapy to preserve normal musculoskeletal function (GUIDELINES FOR THE MANAGEMENT OF HEMOPHILIA, 2^{nd} edition. Prepared by the Treatment Guidelines Working Group, on behalf of the World Federation of Hemophilia (WFH)). A single dose of rIX-FP is capable of maintain FIX activity above 1% for 14 days and beyond.

As is evident from Table 1a and 1b below, rIX-FP also has advantageous PK properties compared to the known rFIX-Fc product.

TABLE 1a
Time dependent FIX activity of a single dose of 50 IU/kg rIX-FP,
50 IU/kg rFIX-Fc, and 100 IU/kg rFIX-Fc.

| | Days | Days | Days | |
|---|---|---|---|---|
| | when FIX | when FIX | when FIX | |
| | activity | activity | activity | |
| | reach | reach | reach | |
| | 1% above | 3% above | 5% above | |
| | baseline | baseline | baseline | |
| 50 IU/kg | | | 14 | Blood. Sep. 20, 2012, |
| rIX-FP | | | | 120(12): 2405-2411 |
| 50 IU/kg | 11.2 | 5.1 | | N Engl J Med. 2013 Dec. |
| rFIX-Fc | | | | 12;369(24):2313-23. doi: |
| | | | | 10.1056/NEJMoa1305074. |
| | | | | Epub 2013 Dec. 4. |
| 100 IU/kg | 12.3 | 8.53 | | Blood. Jan. 19, 2012. |
| rFTX-Fc | | | | 119(3):666-672 |

TABLE 1b
Time dependent FIX activity of a single dose of
50 IU/kg rIX-FP and 50 IU/kg rFIX-Fc.

| | Day 7 mean | Day 14 mean |
|---|---|---|
| | FIX activity | FIX activity |
| 50 IU/kg rIX-FP | 13.41 | 5.54 |
| 50 IU/kg rFIX-Fc | 2.47 | Not tested or no |
| | | data reported |

The ability of FIX-FP to provide such a high FIX activity level at day 7 and continued FIX activity at day 14 and beyond was unexpected. As noted above, rIX-FP has surprisingly so far been the only product dosed 50 IU/kg once every 7 days or longer that maintains a trough of 3% or higher in all patients, including children.

Moreover, a study with a 100 IU/kg rIX-FP dosing regimen was carried out with five patients with severe and moderate severe hemophilia B. The FIX activity (IU/dL in %) was measured, after 0 h, 0.5 b, 72 h (3 days), 168 h (7 days), 336 h (14 days), and 504 h (21 days). The prophylaxis regimen showed a FIX activity of at least 4.1% at day 21-post 100 IU/kg rIX-PP injection. The results are summarized in the Table 8.

The longer half-life and higher specific activity of rIX-FP compared to other known FIX products surprisingly allows for prophylactic treatment of hemophilia with dosing intervals that are significantly longer than suggested by the prior art for rFIX (e.g., Benefix®) and rFIX-Fc. Therefore, rIX-FP has an advantage over rFIX-Fc, as rIX-FP has improved PK parameters compared to rFIX-Fc.

The higher activity of the fusion protein means that less protein is administered, which is less likely to be immunogenic since less host cell proteins are administered, meaning a lower risk of local reactions at the site of injection. The decreased frequency of injections reduces the risk of infections, discomfort for patients, and the number of required visits to a medical professional. These advantages will positively affect patient compliance and thus the effectiveness of prophylactic therapy for hemophilia.

Due to its advantageous properties, rIX-PP has a lower FIX product consumption, such as the dosing regimens disclosed herein. In preferred embodiments, the plasma level of the FIX is maintained at a trough of at least about 0.5%, or at least about 1%, or at least about 2%, or at least about 3%, at least about 4% or at least about 5% above baseline for the entire dosing interval, preferably between 5 and 15% above baseline for the entire dosing interval.

Human FIX

Human FIX, one member of the group of vitamin K-dependent polypeptides, is a single-chain glycoprotein with, a molecular weight of 57 kDa, which is secreted by liver cells into the blood stream as an inactive zymogen of 415 amino acids. It contains 12 γ-carboxy-glutamic acid residues localized in the N-terminal Gla-domain of the polypeptide. The Gla residues require vitamin K for their biosynthesis. Following the Gla domain there are two epidermal growth factor domains, an activation peptide, and a trypsin-type serine protease domain. Further posttranslational modifications of FIX encompass hydroxylation (Asp 64), N- (Asn157 and Asn167) as well as O-type glycosylation (Ser53, Ser61, Thr159, Thr169, and Thr172), sulfation (Tyr155), and phosphorylation (Ser158).

FIX is convened to its active form, Factor IXa, by proteolysis of the activation peptide at Arg145-Ala146 and Arg180-Val181 leading to the formation of two polypeptide chains, an N-terminal light chain (18 kDa) and a C-terminal heavy chain (28 kDa), which are held together by one disulfide bridge. Activation cleavage of Factor IX can be achieved in vitro e.g., by Factor XIa or Factor VIIa/TF. Factor IX is present in human plasma in a concentration of 5-10 μg/ml. Terminal plasma half-life of Factor IX in humans was found to be about 15 to 1& hours (White G C et al. 1997. Recombinant Factor IX. Thromb Haemost. 78: 261-265; Ewenstein B M et al. 2002. Pharmacokinetic analysis of plasma-derived and recombinant F IX concentrates in previously treated patients with moderate or severe hemophilia B. Transfusion 42:190-197).

Half-Life Enhancing Polypeptide (HLEP)

Albumin, albumin family members and immunoglobulins and their fragments or derivatives have been described above as examples of half-life enhancing polypeptides (HLEPs). The terms “human serum albumin” (HSA) and “human albumin” (HA) are used interchangeably in this application. The terms “albumin” and “serum albumin” are broader, and encompass human serum albumin (and fragments and variants thereof) as well as albumin from other species (and fragments and variants thereof).

As used herein, “albumin” refers collectively to albumin polypeptide or amino acid sequence, or an albumin fragment or variant having one or more functional activities (e.g., biological activities) of albumin. In particular. “albumin” refers to human albumin or fragments thereof, especially the mature form of human albumin. For example, albumin can have a sequence or variant thereof, as described in US2008260755A1, which is herein incorporated by reference in its entirety. The albumin portion of the albumin fusion proteins may comprise the full length of the HA sequence, or may include one or more fragments thereof that are capable of stabilizing or prolonging the therapeutic activity. Suck fragments may be of 10 or more amino acids in length or may include about 15, 20, 25, 30, 50, or more contiguous amino acids from the HA sequence or may include part or all of specific domains of HA.

The albumin portion of the albumin fusion proteins of the invention may be a variant of normal HA, either natural or artificial. The therapeutic polypeptide portion of the fusion proteins of the invention may also be variants of the corresponding therapeutic polypeptides as described herein. The term “variants” includes insertions, deletions, and substitutions, either conservative or non-conservative, either natural or artificial, where such changes do not substantially alter the active site, or active domain that confers the therapeutic activities of the therapeutic polypeptides, as described in US2008260755A1, which is herein incorporate by reference in its entirety.

IgG and IgG-fragments may also be used as HLEPs, as long as the HLEP fragments provide a half-life extension of at least 25% as compared to the non-fused coagulation factor. The therapeutic polypeptide portion may be connected to the IgG or the IgG fragments via a linker, preferably a cleavable linker that allows high molar specific activities of the fusion protein, preferably a cleavable linker which is cleavable by proteases involved in coagulation that allow high molar specific activities of the fusion protein at the time coagulation is activated. WO2004/101740 discloses FIX-Fc fusion proteins, and is herein incorporated by reference in its entirety. If these FIX-Fc fusion proteins would have a cleavable linker, they would be comparable to the fusion proteins of the invention.

The invention specifically relates to fusion proteins comprising linking a coagulation factor or fragment or variant thereof to the N- or C-terminus of a HLEP or fragment or variant thereof such that an intervening cleavable peptide linker is introduced between the therapeutic polypeptide and the HLEP such that the fusion protein formed has an increased in vivo half-life compared to the coagulation factor which has not been linked to a HLEP and that the fusion protein has an at least 25% higher molar specific activity compared to the corresponding fusion protein with non-cleavable linker in at least one of the different coagulation-related assays available. It is preferable that the rIX is at the N-terminus of the HLEP fragment or variant thereof.

“Factor IX” within the above definition includes polypeptides that have the natural amino acid sequence including any natural polymorphisms. It also includes polypeptides with a slightly modified amino acid sequence, for instance, a modified N-terminal or C-terminal end including terminal amino acid deletions or additions, as long as those polypeptides substantially retain the activity of the respective therapeutic polypeptide. Variants included differ in one or more amino acid residues from the wild type sequence. Examples of such differences may include truncation of the N- and/or C-terminus by one or more amino acid residues (e.g., preferably 1 to 30 amino acid residues), or addition of one or more extra residues at the N- and/or C-terminus, as well as conservative amino acid substitutions, i.e., substitutions performed within groups of amino acids with similar characteristics, e.g. (1) small amino acids, (2) acidic amino acids, (3) polar amino acids, (4) basic amino acids, (5)hydrophobic amino acids, and (6) aromatic amino acids. Examples of such conservative substitutions are shown in the following table.

TABLE 2
Conservative substitutions of amino acids.

| | | | | |
|---|---|---|---|---|
| (1) | Alanine | Glycine | | |
| (2) | Aspartic acid | Glutamic acid | | |
| (3a) | Asparagine | Glutamine | | |
| (3b) | Serine | Threonine | | |
| (4) | Arginine | Histidine | Lysine | |
| (5) | Isoleucine | Leucine | Methionine | Valine |
| (6) | Phenylalanine | Tyrosine | Tryptophane | |

The in vivo half-life of the fusion proteins of the invention, in general determined as terminal half-life or β-half-life, is usually at least about 25%, preferably at least about 50%, and more preferably more than 100% higher than the in vivo half-life of the non-fused polypeptide.

The fusion proteins of the present invention have at least a 25%, preferably at least a 50%, more preferably an at least 100% increased molar specific activity compared to the corresponding fusion proteins without cleavable linkers.

The molar specific activity (pr molar specific coagulation-related activity as considered here in particular) in this regard is defined as the activity expressed per mole (or e.g. nmole) of the therapeutic polypeptide or therapeutic fusion protein of interest. Calculation of the molar specific activity allows a direct comparison of the activity of the different constructs which are not affected by the different molecular weights or optical densities of the polypeptides studied. The molar specific activity may be calculated as exemplified in Table 3 below for FIX and a FIX-FP fusion protein.

TABLE 3
Calculation of molar specific activity as shown for a purified FIX-HSA fusion protein

| | | | | Molar optical | Calculation of molar |
|---|---|---|---|---|---|
| | | | Activity/Vol/OD₂₈₀ | density (OD₍₂₈₀₎ | specific activity |
| Product | OD_{(280 nm, 1%)} | MW | (IU/L/OD₂₈₀) | at 1 mol/L) | (IU/mol) |
| FIX | 13.3 ¹⁾ | 57 000 | determined for | 75810 (=MW × | =(Activity/Vol/OD₂₈₀) × |
| | | | product | OD_{(280, 1%)}/10) | (OD₂₈₀ at 1 mol/L) |
| Albumin | 5.7 ²⁾ | 66 300 | | 37791 (=MW × | |
| | | | | OD_{(280, 1%)}/10) | |
| FIX-FP | | | determined for | 113601 (=sum | =(Activity/Vol/OD₂₈₀) × |
| | | | product | of molar optical | (OD₂₈₀ at 1 mol/L) |
| | | | | density of FIX | |
| | | | | and albumin) | |

¹⁾ R.G. Di Scipio et at., Biochem.16:6698-706 (1977)
²⁾ C. Chaudhury et al. J. Exp. Med. 197(3):315-322 (2003)

In order to determine a molar specific coagulation-related activity, any assay may be used that determines enzymatic or cofactor activities that are relevant to the coagulation process.

Therefore “coagulation-related assays” in the sense of the invention is any assay which determines enzymatic or cofactor activities that are of relevance in the coagulation process or that is able to determine that either the intrinsic or the extrinsic coagulation cascade has been activated. The “coagulation-related” assay thus may be direct coagulation assays like aPTT, PT, or the thrombin generation assays. However, other assays like, e.g., chromogenic assays applied for specific coagulation factors am also included. Examples for such assays or corresponding reagents are Pathromtin® SL (aPT assay, Dade Behring) or Thromborel® S (Prothrombin time assay, Dade Behring) with corresponding coagulation factor deficient plasma (Dade Behring), Thrombin generation assay kits (Technoclone, Thrombinoscope) using e.g. coagulation factor deficient plasma, chromogenic assays like Biophen Factor IX (Hyphen BioMed), Staclot® FVIIa-rTF (Roche Diagnostics GmbH), Coatest® Factor VIII:C/4 (Chromogenix), or others.

For purposes of this invention, an increase in any one of the above assays or an equivalent coagulation-related assay is considered to show an increase in molar specific activity. For example, a 25% increase refers to a 25% increase in any of the above or an equivalent assay.

To determine whether therapeutic fusion proteins fall within the scope of the present invention, the standard against which the molar specific activity of these proteins is compared is a construct in which the respective coagulation factor and the respective HLEP arm linked by a non-cleavable linker having the amino acid sequence GGGGGGV (SEQ ID NO: 4).

For FIX, aPT assays are often used for determination of coagulation activity. Such a coagulation assay (aPTT assay) is described in example 4 In more detail. However, other coagulation-related assays or assay principles may be applied to determine molar specific activity for FIX.

Although it Is desirable to have a high in vivo recovery and a long half-life for a non-activated coagulation factor, it is advantageous to limit, the half-life of a coagulation factor after its activation or the activation of its co-factor in order to avoid a prothrombotic risk. Therefore, after the coagulation process has been initiated, the half-life of the active coagulation factor should again be reduced. This can either be achieved by enhancing inactivation in a coagulation-related mode or by elimination of the coagulation factor.

Inactivation according to the present invention means the decrease of activity of the therapeutic polypeptide which can be caused, for example, by a complex formation of a coagulation factor and an inhibitor of the corresponding coagulation factor or by further proteolytic cleavage as known, e.g., in the case of FVIII and FV.

The inactivation rate of an activated therapeutic fusion protein is defined as the rate the activity is declining, e.g., by reaction with inhibitors or by proteolytic inactivation. The inactivation rate may be measured by following the molar specific activity of the activated coagulation factor over time in the presence of physiologic amounts of inhibitors of this coagulation factor.

Alternatively, the inactivation rate may be determined after administration of the activated product to an animal followed by testing of plasma samples at an appropriate time frame using activity and antigen assays.

When for therapeutic fusion proteins a determination is needed whether these proteins fall within the scope of the present invention, the standard against which the inactivation rate of these therapeutic proteins is compared to, is a construct in which the respective coagulation factor and the respective HLEP are joined by a non-cleavable linker having the amino acid sequence GGGGGGV (SEQ ID NO: 4).

The elimination rate of an activated therapeutic fusion protein is defined as the rate the polypeptide is eliminated from the circulation of humans or animals. The elimination rate may be determined by measuring the pharmacokinetics of the activated, therapeutic fusion protein after intravenous administration. Using an antigen assay, the elimination by direct removal from the circulation can be determined. Using an activity assay in addition, a specific removal and inactivation rate may be determined.

When for therapeutic fusion proteins a determination is needed whether these proteins fall within the scope of the present invention, the standard against which the elimination rate of these proteins is compared to, is a construct in which the respective coagulation factor and the respective HLEP are joined by the non-cleavable linker having the amino acid sequence GGGGGGV (SEQ ID NO: 4).

According to this invention, the therapeutic polypeptide moiety is coupled to the HLEP moiety by a cleavable peptide linker. The linker should be non-immunogenic and should be flexible enough to allow cleavage by proteases.

The cleavable linker preferably comprises a sequence derived from

a) the therapeutic polypeptide to be administered itself if it contains proteolytic cleavage sites that are proteolytically cleaved during activation of the therapeutic polypeptide,

b) a substrate polypeptide of this therapeutic polypeptide, or

c) a substrate polypeptide cleaved by a protease which is activated or formed by the direct or indirect involvement of the therapeutic polypeptide.

The linker region in a more preferred embodiment comprises a sequence of the therapeutic polypeptide to be applied, which should result in a decreased risk of neoantigenic properties of the expressed fusion protein.

In a preferred embodiment, the HLEP is albumin. In this case the linker sequence is either derived from the sequences of the activation regions of FIX, front the cleavage region of any substrate of FIX like FX or FVII or from the cleavage region of any substrate polypeptide that is cleaved by a protease in whose activation FIXa is involved.

In a highly preferred embodiment the linker peptide is derived front FIX itself. In another preferred embodiment the linker peptide is derived from FX or FVII. In another preferred embodiment the linker sequence comprises two cleavage sequences that can be cleaved by FXIa or FVIIa/TF, two physiologically relevant activators of FIX.

Variants and fragments of the described linkers are also encompassed in the present invention as long as the linker can still be cleaved by the protease or the proteases that cleave the linkers. The term “variants” includes insertions, deletions and substitutions, either conservative or non-conservative.

Pharmaceutical Compositions and Modes of Administration

The fusion proteins of the invention Cat be incorporated into pharmaceutical compositions suitable for administration. Such compositions typically comprise the protein and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of mutes of administration include parenteral, e.g., intravenous, intradermal, subcutaneous, oral (e.g., inhalation), transdermal (topical); transmucosal, and rectal administration. Solutions or suspensions used for parenteral, intradermal, or subcutaneous, application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases; such as hydrochloric acid of sodium hydroxide. The parenteral preparation can be enclosed in ampoules, disposable syringes or multiple dose vials made of glass of plastic. Administration as an intravenous injection is the preferred route of administration.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringe ability exists. It must be stable under the conditions of manufacture and storage and must be preserved against the contaminating action of microorganisms such as bacteria and fungi. The carrier can be a solvent or dispersion medium containing, for examples, water, ethanol, polyol (for example, glyceol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol sodium chloride in the composition, Prolonged absorption of the injectable compositions can be brought, about by including in the composition an agent which delays absorption, for example, aluminum monostearate and gelatin.

Sterile injectable solutions can be prepared by incorporating the active compound (e.g., rIX-FP) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

The formulations for injection can contain the Factor IX (FIX) fusion protein (e.g., rFIX-FP) in a therapeutically effective amount, which amount may be determined by the skilled person. In particular, the Factor IX (FIX) fusion protein (e.g., rFIX-FP) may administered at a concentration of about 0.100 to 400 IU/ml. For example, the fusion protein may be provided for administration at a concentration of about 100, 200 or 400 IU/ml. The fusion protein may be provided for administration at higher concentrations such as 600 IU/ml and 1200 IU/ml.

It is especially advantageous to formulate pharmaceutical compositions, such as compositions for injection, in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms are dictated by and directly dependent on the unique characteristics of the active compound and the particular therapeutic effect to be achieved.

The pharmaceutical compositions can be included in a container, pack, or dispenser together with instructions for administration.

### DESCRIPTION OF THE FIGURES

FIG. 1: Schematic of the advantageous properties of rIX-FP

FIG. 2: Amino Acid sequence of mature rIX-EP (SEQ ID NO: 1). FIX is shown in as 1-416, the linker sequence is in bold and underlined (aa416-433) and the albumin sequence is shown in aa 434-1018.

FIG. 3: Schematic diagram of the trial design in Example 1, including time periods, duration and subject flow, PK, pharmacokinetics.

FIG. 4: Schematic diagram showing the flow and disposition of patients in the trial of Example 1.

FIG. 5: (A) The number of rIX-FP infusions to achieve hemostasis for all treated bleeds, (B) The time between the start of a bleed to the first infusion of rIX-FP, for all treated bleeds.

FIG. 6. The mean annualized bleeding rate for spontaneous bleeds are shown in (A) and for all bleeds are shown in (B). The annualized bleeding rate for each patient was calculated as the number of bleeds during the time in the treatment period of the study in days, divided by 365.25. The historical bleeding rate for each patient was the number of bleeds in the 12 month period prior to study entry PT, prophylaxis treatment; ODT, on-demand treatment.

FIG. 7: PK results for 100 IU/kg rIX-PP once every 21 days (simulated FIX activity vs. time).

FIG. 8: Mean values of rIX-FP activity over a time, following an injection with 100 IU/kg rIX-FP.

FIG. 9: Mean values of rIX-FP activity over a time, following an injection with 100 IU/kg rIX-FP, or an injection with 50 IU/kg rIX-FP, and mean values of previous FIX (e.g., Benefix®), following an injection with 50 IU/kg FIX.

FIG. 10: Median or average FIX activity levels (solid line) and the boundaries characterizing the lowest 5% and the highest 5% of the FIX activity values for dosing regimens such as 25 IU/kg weekly, 50 IU/kg every 10 days and every 14 days, and 75 IU/kg every 14 and every 21 days. The trough levels for the median and for the lowest and highest 5% are shown in Table 9b.

FIG. 11: Flow chart of the study arms 1 and 2 of the phase II/III study for evaluating the efficacy, pharmacokinetics and safety of rIX-FP.

FIG. 12: PK results for 33.33, 50, and 75 IU/kg rIX-FP once every 7, 10, and 14 days, respectively (simulated FIX activity vs. time), and comparison to PK results for 50, 100, and 100 (U/kg Alprolix™ once every 7, 10, and 14 days, respectively (simulated FIX activity vs. time).

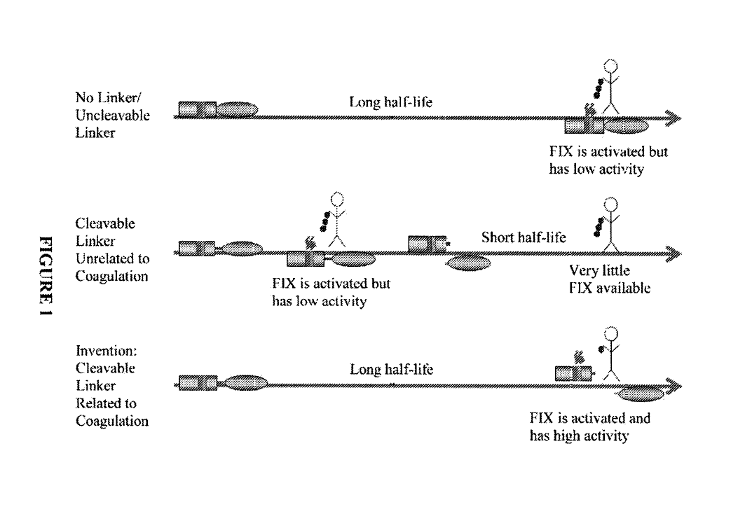

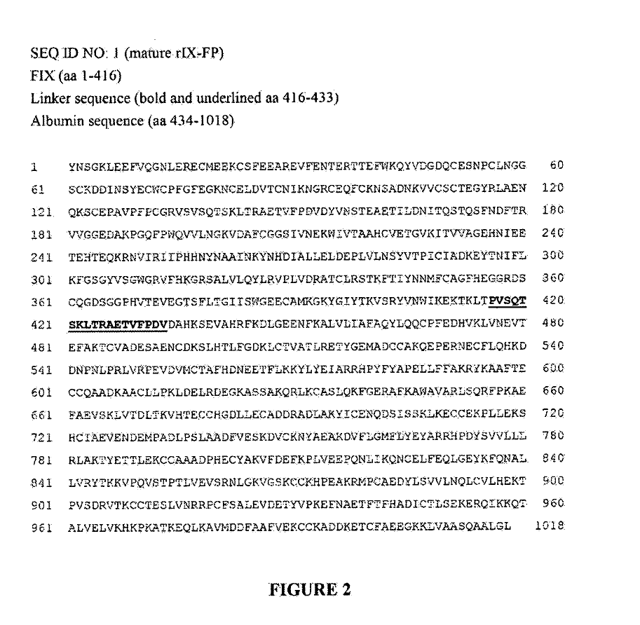

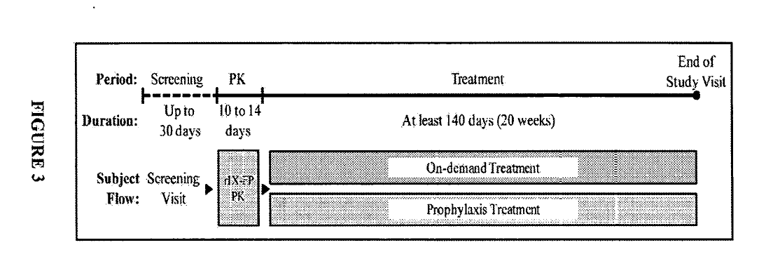

Having now described the present invention in detail, the same will be more clearly understood by reference to the following examples, which are Included herewith for purposes of illustration only and are not intended to be limiting of the invention. All patents and publications referred to herein are expressly incorporated by reference.

### EXAMPLES

### Example 1

### Phase I/II Open-Label Trial of Safety and Efficacy of a Novel Recombinant Fusion Protein Linking Coagulation Factor IX with Albumin (rIX-FP) in Hemophilia B Patients

The present trial aimed to evaluate the efficacy of rIX-FP for the prevention of bleeding episodes during once weekly prophylaxis and to assess the hemostatic efficacy for the treatment of bleeding, in addition to assessing safety and pharmacokinetics (PK) of rIX-FP.

Patients and Methods

Patients

The criteria for subject selection were based on the draft Guideline on the clinical investigation of recombinant and human plasma-derived factor IX products by the Committee for medicinal products for human use (European Medicines Agency. Committee for medicinal products for human use (CHMP), Guideline in the Clinical Investigation of Recombinant and Human Plasma-Derived Factor IX Products. 2009. CHMP/BPWP/144552/2009. Available at http://www.ema.europeu/docs/en_GB/document_library/Scientific_guideline/2009/09/WC500003634.pdf). Patients were previously treated (≧150 exposure days to FIX products) males with hemophilia B (FIX activity ≦2%) and aged 12 to 65 years. Patients with a history of neutralizing antibodies (inhibitors) to FIX, a CD4+ lymphocyte count <200/mm³ (if HIV positive) or with a coagulation disorder other than hemophilia B were excluded from participation. Patients were recruited from 2 sites in 2 countries (Israel and Bulgaria). All patients (or the patient's parents or legally acceptable representative) provided written informed consent prior to any trial-related activities. The study was approved by independent ethics committees, and was conducted in accordance with GCP and the Declaration of Helsinki. The trial was registered at www.clinicaltrials.gov under identifier NCT01361126.

Trial Design

This trial was a prospective, open-label study to evaluate the safety, pharmacokinetics and efficacy of rIX-FP, which is being developed for the prophylaxis and treatment of bleeding episodes in patients with congenital Factor IX (FIX) deficiency (hemophilia B). The study consisted of a 10 to 14 day evaluation of rIX-FP PK, and an 11-month safety and efficacy evaluation period with subjects receiving weekly prophylaxis treatment, and a 3 to 5 month safety and efficacy evaluation period in subjects receiving on-demand treatment (see FIG. 3). Subjects receiving weekly prophylactic treatment were initially treated with 30±5 IU/kg. The dose could be adjusted based on bleeding phenotype, physical activity level, and clinical outcome, while maintaining a 7-day treatment interval and individualized through FIX activity level. All bleeding events which occurred during the active treatment period of the study were treated with rIX-FP; bleeding events which occurred during screening or during the pharmacokinetic assessment period could be treated with the subject's previous product at the discretion of the investigator. For the on-demand treatment of a bleeding event, the dose was based upon the subject's PK profile, WFH guidelines and local standard of care, with a minimum dose of 25 IU/kg rIX-FP. All subjects self-administered rIX-FP treatment for both routine prophylaxis and the treatment of bleeding events, treatments and bleeding events were recorded by subjects in an electronic diary.

Trial Objectives and Endpoint

The primary objective of the study was to evaluate the long term safety of intravenous injections of rIX-FP. Safety was evaluated by the nature and incidence of adverse events, changes in laboratory values, and the development of inhibitors or non-neutralizing antibodies against rIX-FP.

The secondary objectives of the study were to evaluate the PK parameters following a single IV dose of 25 IU/kg rIX-FP, the clinical response of weekly routine prophylaxis with, rIX-FP with respect to the prevention of bleeding episodes and clinical response of bleeding episodes treated with rIX-PP.

Analytical Methods

FIX activity was measured using a validated one-stage clotting method. Briefly, the test samples were mixed with equal amounts of FIX depleted plasma and tested by in vitro determination of activated partial thromboplastin time (aPTT) using. Pathromtin SL (Siemens Healthcare Diagnostics, Marburg) as activator reagent, rIX-FP activity determination was performed using the Behring Coagulation System (BCS). The results were interpreted using a reference curve, which was prepared from standard human plasma (SHPL) calibrated by the manufacturer against WHO standard (International Blood Coagulation Factors II, VII, IX, X, Human, Plasma) for FIX, and the results are reported in percent of norm or International Units.

Inhibitors were titrated by the Bethesda method according to the Nijmegen modification, a coagulation assay based on in vitro determination of aPTT in human citrated plasma. A result ≧0.6 Bethesda Units (BU) was defined as a positive result.

A tiered approach to immunogenicity testing for rIX-FP was employed during the study. Antibodies to rIX-FP were tested in all patients before rIX-FP exposure and 4 weeks after exposure. A direct binding ELISA assay was employed to detect antibodies against rIX-FP; if a positive signal was obtained, the plasma sample was re-tested in a separate direct binding ELISA assay to confirm the specific antibody signal and to discriminate between antibodies against plasma-derived FIX, recombinant FIX (BeneFIX) and albumin.

The analyses of FIX activity, FIX antigen, inhibitors and antibodies against rIX-FP were performed in the central laboratory at CSL Behring, Marburg, Germany.

Pharmacokinetic Analysis and Statistical Methods

Following a 4-day washout, blood samples for measurement of FIX activity for PK analysis were collected prior to dosing rIX-FP and at 30 minutes, 3, 24, 48, 72, 120, 168, 240 and 336 hours after infusion. All PK parameters were calculated using the actual collection times, according to ISTH recommendations. Patients who received a FIX product for the treatment of a bleed during the PK sampling period or who did not have a sufficient number of analyzable PK samples were excluded from the PK analysis. The PK analysis was performed by standard non-compartmental analysis (NCA) using WinNonlin® software (Pharsight). PK parameters included: ara under the curve to the last sample with quantifiable drug concentration (AUC₀₋ₜ); area under the curve from the time of dosing extrapolated to infinity, based on the last observed FIX concentration (AUC_{0-inf}); incremental recovery (IR₀₋₃₀ₘᵢₙ) according to the formula C₃₀ₘᵢₙ (IU/dL)/Dose (IU/kg); terminal half-life (t_{1/2}); total body clearance, normalized to body weight (CL).

The safety endpoints were summarized using descriptive statistics, including all patients exposed to rIX-FP (safety population).

Efficacy

The numbers of infusions to achieve hemostasis were tabulated to determine the efficacy of rIX-FP for the treatment of bleeding events. In addition, the investigators rated the efficacy of the treatment of bleeding events on a four-point scale, which took into account both the number of infusions taken by the subject as well as the subject-reported pain relief after treatment.

The efficacy of routine prophylaxis was measured by the consumption of rIX-FP and by the number of spontaneous bleeding events that occurred while on: prophylaxis, which is displayed as an annualized bleeding rate.

Drug Product

rIX-FP is a single chain glycoprotein with a molecular weight of approximately 125,000 Da, synthesized in CHO cells. The manufacturing and formulation do not include the addition of excipients from animal or human origin (Metzner H J, Weimer T, Kronthaler U, et al. Genetic fusion, to albumin improves the pharmacokinetic properties of Factor IX. Thrombosis and Homeostasis 2009; 102: 634-44). rIX-FP is a highly purified recombinant fusion protein linking recombinant human coagulation FIX with recombinant human albumin by a short, cleavable linker derived from an endogenous FIX sequence involved in FIX activation. The linker is cleaved from the fusion protein by the same enzymes, e.g. coagulation Factor XIa or Factor VIIa/Tissue Factor, which activate FIX during the process of blood coagulation, removing the albumin moiety, rIX-FP was supplied as a lyophilized sterile formation intended for IV injection in single-use vials of 500 and 1000 IU/vial, and was reconstituted with 2.5 mL sterile water for injection.

Results

Patient Characteristics

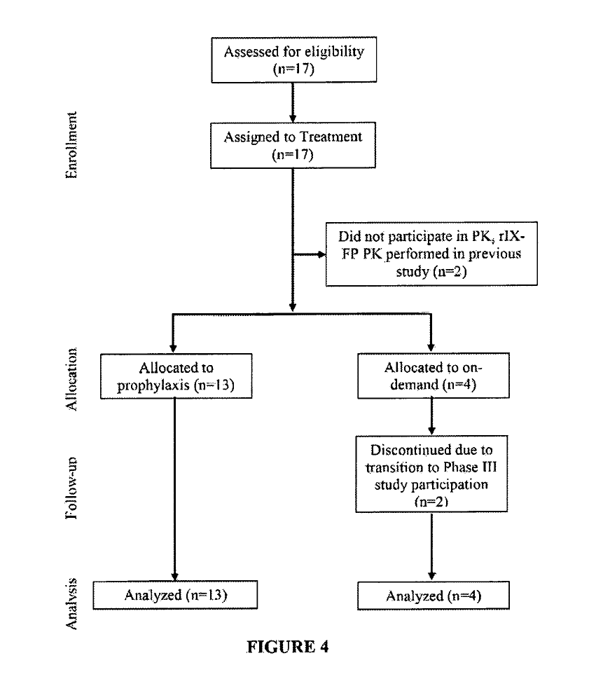

Seventeen study subjects from hemophilia treatment centers in Israel and Bulgaria were screened and all were enrolled in the study. All subjects were Caucasian and non-Hispanic, and their ages ranged from 13 to 46 years (mean age 26 yeas). All 13 subjects enrolled in Israel received weekly prophylaxis treatment with rIX-FP for the duration of the study (range 37-48 weeks), and all 4 subjects enrolled in Bulgaria received on-demand treatment for bleeding episodes with rIX-FP for the duration of the study (range 15-22 weeks). The subject disposition is outlined in FIG. 4.

At screening, 5 (29%) subjects were hepatitis C positive, and 1 (6%) subject was hepatitis B positive and no subject was HIV positive. Almost half of the subjects (8/17) reported a musculoskeletal disorder, including hemophilic arthropathy and chronic synovitis. Five subjects had a previously documented history of Gilbert's syndrome, which was evidenced by high bilirubin levels at baseline. Overall, the prophylaxis subjects were younger than the on-demand subjects, and included three subjects younger than 18 years old. The prophylaxis subjects had fewer chronic hepatitis infections than the on-demand subjects, and reported less joint damage (Table 4).

TABLE 4
Patient Demographics and Medical History

| | Prophylaxis | | On Demand | | | |
|---|---|---|---|---|---|---|
| | Treatment | | Treatment | | Total | |
| | N = 13 | | N = 4 | | N = 17 | |
| Age, years, mean (min-max) | 23.2 | (13-42) | 35.8 | (27-46) | 26.1 | (13-46) |
| <18 years, n (%) | 3 | (23.1) | 0 | | 3 | (17.6) |
| Weight, kg, mean (min-max) | 64.1 | (36.0-83.8) | 75.7 | (62.4-93.0) | 66.8 | (36.0, 93.0) |
| Race | | | | | | |
| White | 13 | (100.0) | 4 | (100.0) | 17 | (100.0) |
| Previous Exposure Days to FIX, | 861.9 | (353.61) | 662.5 | (131.5.0) | 815.0 | (323.46) |
| mean (SD) | | | | | | |
| Total Bleeds 12 months prior to | 14.0 | (17.97) | 27.0 | (3.37) | 17.1 | (16.63) |
| study entry, mean (SD) | | | | | | |
| Spontaneous Bleeds 12 months | 9.2 | (14.73) | 27.0 | (3.37) | 13.4 | (15.02) |
| prior to study entry, mean (SD) | | | | | | |
| Prior treatment | | | | | | |
| Prophylaxis, n (%) | 10 | (76.9) | 0 | | 10 | (58.8) |
| On-demand, n (%) | 3 | (23.1) | 4 | (100.0) | 7 | (41.2) |
| HIV, n (%) | 0 | | 0 | | 0 | |
| HBV, n (%) | 0 | | 1 | (25.0) | 1 | (5.9) |
| HCV, n (%) | 3 | (23.1) | 2 | (50.0) | 5 | (29.4) |
| Haemophilic arthropathy, n (%) | 5 | (38.5) | 4 | (100.0) | 5 | (29.4) |

| | | | | | |
|---|---|---|---|---|---|
| Gilbert's syndrome, n (%) | 5 | (38.5) | 0 | 5 | (29.4) |
| Synovitis, n (%) | 3 | (23.1) | 0 | 3 | (17.6) |

Min, minimum; Max, maximum; n, number of patients; SD, standard deviation.

Subjects in the safety population had an estimated mean of 815 exposure days (EDs) to FIX products prior to study entry (range 415-1450). In the study prophylaxis treatment group, 3 subjects had been receiving on-demand treatment prior to study entry, and 10 subjects were receiving prophylaxis treatment with FIX products (2-3 times per week in 80% of the subjects). Those 3 subjects previously receiving on-demand treatment reported a much higher mean number of total bleeds in the 0.12 months prior to study entry than the prior prophylaxis subjects (43.3 vs. 5.2). Subjects in the on-demand group in the study had a mean of 27 bleeds in the 12 months prior to study entry.

Pharmacokinetics

The pharmacokinetics (PK) of a single dose of 25 IU/kg rIX-FP was assessed at the beginning of the study in 15 subjects who had not previously received rIX-FP. The PK parameters were comparable to those previously reported from the Phase I study (Santagostino E, Negrier C, Klamroth R, Tiede A, Pabinger-Fasching I, Voigt C, et al. Safety and pharmacokinetics of a novel recombinant fusion protein linking coagulation factor IX with albumin (rIX-FP) in hemophilia B patients. Blood 2012; 120: 2405-11), and the mean single-dose pharmacokinetic profile has been previously published (Martinowitz U, Lubetsky A. Phase I/II open-label, multicenter, safety, efficacy and PK study of a recombinant coagulation factor IX albumin fusion protein (rIX-FP) in subjects with hemophilia B. Thrombosis Research 2013; 131S2: S11-S14). A dose of 25 IU/kg rIX-FP had a mean incremental recovery of 1.52 IU/dL per IU/kg and a mean half-life of 94.8 hours. The mean baseline-uncorrected FIX activity at 7, 10 and 14 days were 5.6, 3.9 and 2.9 IU/dL following a single dose of 25 IU/kg rIX-FP.

Dose

All prophylaxis subjects were initially assigned a weekly prophylaxis dose of 25 to 35 IU/kg (mean of 30 IU/kg) rIX-FP as prescribed by the protocol. Over the course of the study, the dose was adjusted by the investigator due to physician decision (69.7%), bleeding (18.2%) or other (12.1%) reasons. The mean dose for weekly prophylaxis was 58.6 IU/kg (range 47.6-75 IU/kg) during the last 12 weeks of the study, aim to prevent not only spontaneous bleed, but also trauma-induced bleed with high trough FIX activity level. The trough levels at these doses were high, with a mean of 36.2% FIX activity at 5 days (122 hours) after dosing.

For on-demand treatment of bleeding episodes, all subjects were initially assigned a dose of 30-35 IU/kg (mean of 33 IU/kg) rIX-FP as prescribed by the protocol. Over the course of the study, the dose was adjusted by the investigator due to physician decision (67.6%), bleeding (21.6%) or other (10.8%) reasons. The mean dose for the treatment of all bleeding episode was 46 IU/kg, with prophylaxis subjects assigned a higher dose than on-demand subjects (mean dose 62 IU/kg vs. 28 IU/kg). The on-demand dose assignments were adjusted at the same time and to the same dose as the prophylaxis dose, for subject's convenience, resulting in higher doses in the prophylaxis group.

Safety

rIX-FP was well-tolerated in all subjects. The duration of treatment ranged from 259 to 335 days for prophylaxis subjects, and from 105 to 155 days for on-demand subjects, with subjects receiving prophylaxis participating in the study longer than on-demand subjects (mean 315 days vs 131 days). There were a total of 718 EDs to rIX-FP, with a mean of 51.5 EDs to rIX-FP in the prophylaxis subjects and 12 EDs to rIX-FP in on-demand subjects. Nine prophylaxis subjects achieved at least 50 EDs during the study.

None of the subjects developed inhibitors to FIX or antibodies to rIX-FP following rIX-FP administration. One subject showed transient positive, antibodies to plasma-derived FIX and BeneFIX prior to the first dose of rIX-FP, which resolved by Week 12, at which time this subject was negative for antibodies to all antigens tested. There were no hypersensitivity reactions. There were no significant treatment-emergent findings in any safety-related parameters during the course of the study.

A total of 14 (82.4%) subjects reported 46 treatment-emergent adverse events, none of which were considered related to rIX-FP by the investigator (Table 5).

TABLE 5
Overview of Treatment Emergent Adverse Events

| | rIX-FP, N (%) E | | |
|---|---|---|---|
| | Prophylaxis | On Demand | Total |
| Number of subjects | 13 | 4 | 17 |
| AE Leading to Study | 0 | 0 | 0 |
| Withdrawal | | | |
| Serious AEs (SAEs) | 0 | 0 | 0 |
| Any AEs | 13 (100.0) 45 | 1 (25.0) 1 | 14 (82.4) 46 |
| Severity of AEs | | | |
| Mild | 13 (100.0) 42 | 1 (25.0) 1 | 14 (82.4) 43 |
| Moderate | 2 (15.4) 3 | 0 | 2 (11.8) 3 |
| Severe | 0 | 0 | 0 |
| AEs Related to rIX-FP | 0 | 0 | 0 |

N, number of subjects with adverse events; E, events; AE, adverse events

All AEs were mild or moderate in severity. There were no serious adverse events reported, and there were no withdrawals due to AEs. The most frequent classes of AEs were musculoskeletal disorders (7 (41.2%) subjects, 17 events) and injuries (7 (41.2%) subjects, 9 events).

Efficacy

Treatment of Bleeds.

Seven (53.8%) prophylaxis subjects and 4 (100%) on-demand subjects treated spontaneous bleeding episodes. During the study, a total of 85 bleeding episodes were reported which required treatment with rIX-FP, of which approximately half (54.1%) were spontaneous. Among prophylaxis subjects, all 14 spontaneous bleeds which occurred during the prophylaxis treatment period were in ankle or elbow joints.

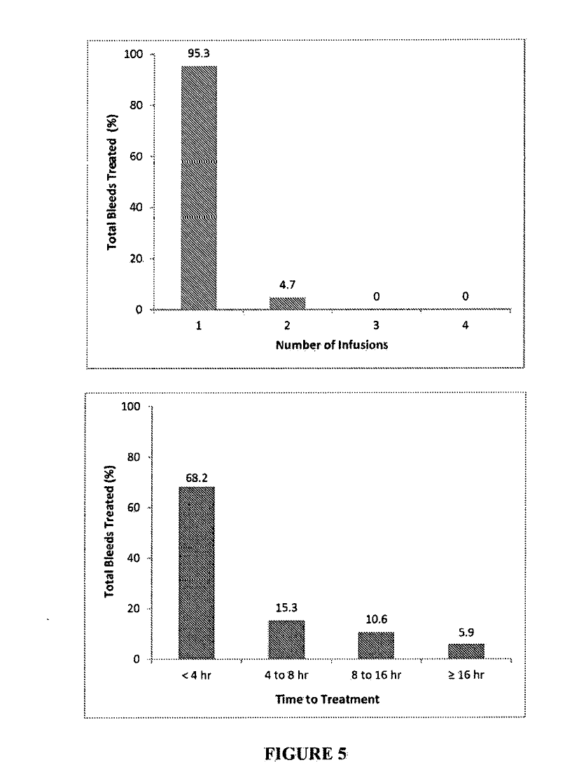

Control of bleeding was assessed in all subjects who experienced a bleeding episode during the study; all bleeding episodes requiring treatment were successfully treated with one (95.3%) or two (4.7%) doses of rIX-FP during the study (FIG. 5A). On demand subjects treated bleeds with a mean dose of 28 IU/kg rIX-FP, and 97.3% of bleeds (37 of 38) were treated with a single dose of rIX-PP. Prophylaxis subjects treated bleeds with a mean dose of 62 IU/kg, and 93.6% of bleeds (44 of 47) were treated with a single dose of rIX-FP. There were 4 bleeds requiring two doses of rIX-FP to achieve hemostasis. An additional 5 bleeds were treated with a single dose of rIX-FP, followed by the scheduled prophylaxis dose of rIX-FP within 30 hours of the treatment dose.

The majority of infusions received a rating of excellent or good (96.5%) by the investigator in response to rIX-FP treatment. There were 3 doses which received a moderate rating; these bleeds all resolved with 1 or 2 infusions, and the time to the first rIX-FP treatment was delayed more than 8 hours after the start of the bleed. While subjects were encouraged to treat a bleed immediately, 16.4% of bleeds were treated more than 8 hours after the start of the hemorrhage, and 5.9% more than 16 hours after the start of the hemorrhage (FIG. 5B).

Routine Prophylaxis.

All thirteen prophylaxis subjects maintained weekly routine prophylaxis with rIX-FP throughout the 11 months study. Approximately half of the prophylaxis subjects (46%, 6 subjects) did not have any spontaneous bleeding events for the duration of the study. Seven prophylaxis subjects reported 14 spontaneous bleeds during the prophylaxis portion of the study and 2 spontaneous bleeds occurred at the end of the pharmacokinetic period. Four of the seven subjects reporting spontaneous bleeds had a history of hemophilic arthropathy or synovitis in the joint; the remaining 3 subjects were all teenagers, two of whom were receiving only on-demand treatment prior to study entry.

Among the 10 subjects who received prophylaxis with a FIX product prior to study entry, 5 received pdFIX and 5 received rFIX on various treatment regimens from once weekly to 3 times a week. Subjects had a mean weekly consumption of 87.7 IU/kg of FIX prior to entering the study, and a mean weekly dose of 58.6 IU/kg of rIX-FP at the end of the study. Mean weekly consumption of rIX-FP was 33% less than the mean weekly consumption of the FIX product used prior to study entry.

Annualized Bleeding Rate.

Overall, prophylaxis subjects reported fewer spontaneous bleeding episodes than the subjects receiving on-demand treatment only. On-demand subjects had a mean of 8.0 spontaneous bleeding episodes during the study, for an annualized bleeding rate (ABR) of 21.74 spontaneous bleeds per year. Prophylaxis subjects had a mean of 1.1 bleeding episodes during the study, for an annualized rate of 1.26 spontaneous bleeds per year. The prophylaxis and on-demand subjects had a similar annualized rate of traumatic bleeding events per year. The mean ABR for total bleeds (spontaneous and traumatic) during the study was 4.35 and 28.8 for prophylaxis and on-demand subjects, respectively.

In the prophylaxis treatment group, ten subjects received prophylaxis regimen prior to study entry. These 10 subjects reported an approximately 50% or 30% higher mean spontaneous blooding rate or total bleeding rate, respectively in the 12 months prior to study entry than the ABR with weekly prophylaxis of rIX-FP during the study.

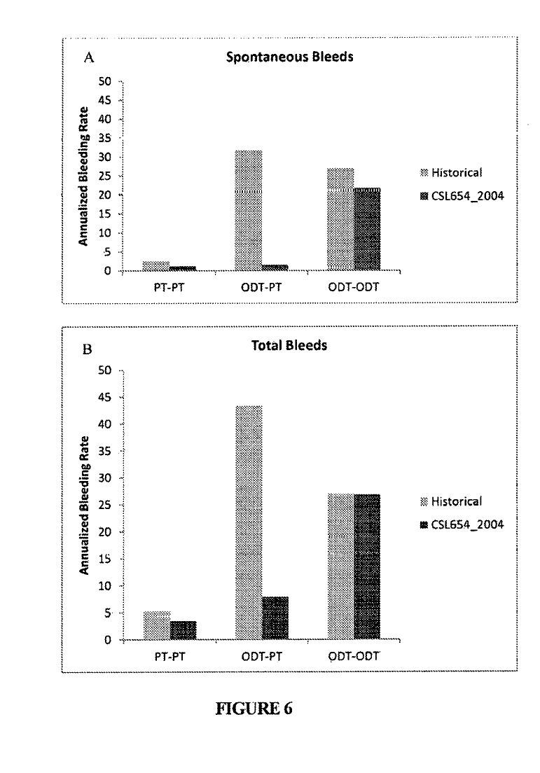

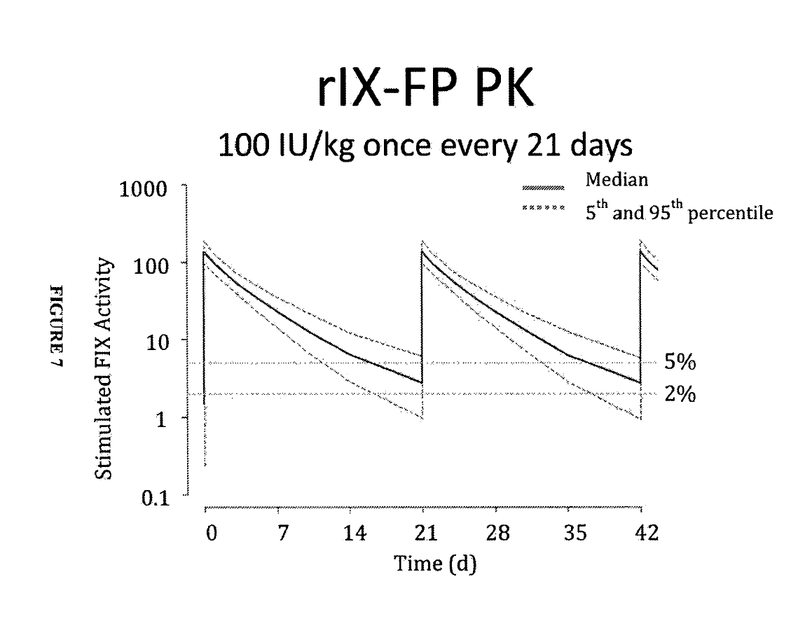

In the prophylaxis treatment group, three subjects received on-demand treatment prior to study entry. These 3 subjects reported a much higher historical mean spontaneous bleeding rate (31.7) and total bleeding rate (43.3) in the 12 months prior to study entry than prophylaxis subjects who received prophylaxis treatment prior to study entry (FIG. 6). After starting weekly prophylaxis with rIX-FP, the mean annualized spontaneous and total bleeding rates were 1.56 and 7.3 per year, respectively. In this subset of subjects, a 95% or 83% reduction in mean annualized spontaneous or total bleeding rate when compared to the historical data.

Discussion

Thirteen patients with severe hemophilia B received weekly prophylaxis treatment with rIX-FP for 11 months, and 9 achieved at least 50 exposure days. In addition, 4 patients received rIX-FP only when experiencing a bleeding event for a period of 3 to 5 months.

Albumin fusion technology has been shown to be a very attractive technology to extend the half-life of coagulation factors, as human albumin is an abundant plasma protein and does not act as a trigger for the immune system. In the present study, there were no hypersensitivity reactions or development of inhibitors to FIX or antibodies to rIX-FP after over 700 repeated exposures to rIX-FP among 17 study subjects.

rIX-FP was very effective in the treatment of bleeding events, with hemostasis achieved after a single dose 95% of the time, and all bleeds effectively treated with one or two doses. This compares favorably to treatment with BeneFIX®, which has been reported to effectively treat 81% of bleeds with a single dose in a clinical trial in previously treated patients (BeneFIX® Package Insert. 2011 November Available at: http://labeling.pfizer.com/showlabeling.aspx?id=492). While there was a large range in the rIX-FP dose used to treat bleeds, there is no apparent correlation between the dose of rIX-FP used and the number of treatments needed to achieve hemostasis. All of the prophylaxis subjects were assigned a high dose of rIX-FP for the treatment of any bleeding event. The dose chosen was, for patient convenience, as the prophylaxis and treatment doses were the same for every prophylaxis patient, as decided by the treating physician. On demand subjects treated bleeds with a mean dose of 28 IU/kg (50% less rIX-PP than the dose used in prophylaxis subjects) and had a similar success rate, with 97.3% of bleeds in on-demand subjects treated with a single dose of rIX-FP. Prophylaxis subjects treated bleeds with a mean dose of 62 IU/kg, and 93.6% of bleeds were treated with a single dose a rIX-FP. The difference in assigned rIX-FP doses for the treatment of bleeds may be the result of multiple factors, including the prophylaxis treatment status, the bleeding phenotype of the patient and local standard of care, but the lower dose of rIX-FP was equally effective for the treatment of bleeding events.

All 13 prophylaxis subjects maintained weekly treatment interval for prophylaxis with rIX-FP for 11 months during the study. There was excellent, treatment compliance with the majority of subjects taking their prescribed dose as scheduled. The annualized bleeding rate of these subjects on weekly prophylaxis was less than their previous bleeding rate when receiving prophylaxis 1, 2 or 3 times weekly with plasma-derived or recombinant FIX. During the study, 6 subjects reported no spontaneous bleeding episodes, 5 of whom had no spontaneous bleeding episodes during the 12 months prior to the study entry. There were 3 subjects who were not receiving prophylaxis treatment prior to study entry; these subjects had 83% reduction in the annualized bleeding rate compared to their reported annualized bleeding rate while on on-demand treatment. Remarkably, 1 of these 3 subjects had no spontaneous bleeding episodes during the study. While this is an extremely small sample size, switching from on-demand to weekly prophylaxis treatment of rIX-FP dramatically reduced the bleeding rate for these subjects.

This proof of concept study demonstrated that a less frequent prophylaxis treatment regimen is possible and effective with the extended half-life provided by recombinant fusion protein linking coagulation FIX with albumin (rIX-FP). In addition, rIX-FP provided effective on-demand treatment for patients with this life-long, debilitating bleeding disorder. The safety profile may make rIX-FP an excellent choice for long-term prophylaxis for hemophilia B patents.

### Example 2

### A Phase 3b Open-Label, Multicenter, Safety and Efficacy Extension Study of a Recombinant Coagulation Factor IX Albumin Fusion Protein (rIX-FP) in Subjects with Hemophilia B

1. Study Overview

A prospective, open-label study to evaluate the long term safety and efficacy of rIX-PP is being developed for the prophylaxis treatment of bleeding episodes in subjects with hemophilia B. The study will include but not limited to study subjects who were enrolled in our prior clinical phase II/III and phase II studies. In addition, subjects requiring major non-emergency surgery who have not previously completed a CSL-sponsored rIX-FP lead-in study may be enrolled. At the end of this study, subjects from the lead-in studies are expected to have accumulated at least 100 rIX-FP exposure days during enrollment in all CSL-sponsored rIX-FP studies.

This extension study consists of a prophylaxis treatment period of approximately 3 years during which subjects will administer rIX-FP as routine prophylaxis. During an initial 6-month period, subjects will receive prophylactic treatment with rIX-FP administered using the following treatment intervals:
Arm 1; Once every 7, 10, or 14 days
Arm 2: Once every 7 days
Arm 3: One every 7 days

After this initial 6-month period, all subjects will receive prophylactic therapy with rIX-FP administered once every 7, 10, 14, or 21 days for an additional approximately 30 months. Subjects transferring to a 21-day treatment interval must be ≧18 years of age. Any subject transferring to a 21-day treatment interval for the first time must have completed at least 6 months of prophylactic treatment with a 14-day treatment interval and must undergo an initial PK evaluation period with a single rIX-FP dose of 100 IU/kg. During the study, a subject may undergo additional rIX-FP PK evaluations at the investigator's discretion or CSL's request.

Subjects in Arm 3 who require major non-emergency surgery Will undergo an initial PK evaluation (100 IU/kg rIX-PP) to determine the incremental recovery and FIX activity. These subjects will them complete the surgery substudy after which they may start the prophylaxis treatment period.

Prophylaxis treatment using a 21-day treatment interval will only be available to subjects ≧18 years of age and who have completed at least 6 months of prophylaxis treatment with a 14-day treatment interval. If subjects do not benefit from a treatment interval of 21 days, they will be able to transfer to the 7, 10, or 14-day treatment interval at any time, at the discretion of the investigator.

This study will document all key safety factors including immunogenicity, thrombogenicity, hypersensitivity, and other AEs in the intended therapeutic population.

Thus, the associated benefit risk assessment of the study is acceptable for subjects enrolled in the study.

2. Study Objectives and Endpoints

2.1 Primary Objective and Endpoints

2.1.1 Primary Objective

The primary objective of this study is to evaluate the safety of rIX-FP as measured by new cases of inhibitors against FIX in subjects with severe hemophilia B.

2.1.2 Primary Endpoints

The total number of subjects, who develop inhibitors against FIX after approximately 3-year participation in this extension study.

2.2 Secondary Objective and Endpoints

2.2.1 Secondary-Objectives

The secondary objectives of the study are:
To evaluate the efficacy of rIX-FP routine prophylaxis when administered at various treatment intervals.
To compare the efficacy of rIX-FP routine prophylaxis between 2 different treatment
   intervals and versus on-demand treatment.
To further evaluate the safety of rIX-FP.

2.2.2. Secondary Endpoints
Annualized bleed rate (ABR) by treatment interval for spontaneous treated and total treated bleeds.
For subjects from a lead-in phase I/III study Arm 2, comparison of the ABR of spontaneous treated bleeds between routine prophylaxis treatment interval of 14 days in this extension study and:
   On-demand only treatment during the lead-in study.
   Prior routine prophylaxis treatment with a treatment interval of 7 days during the lead-in study and this extension study.
Consumption of rIX-FP during routine prophylaxis expressed as IU/kg per month per subject.

2.3 Exploratory Objectives and Endpoints

2.3.1 Exploratory Objectives

Other, exploratory objectives of the study include:
To further evaluate the efficacy of rIX-FP when used for routine prophylaxis at various treatment intervals over a period of up to 3 years.
To evaluate the PK profiles of rIX-FP at a dose of 100 IU/kg.
To describe the QoL of pediatric subjects from lead-in phase III study after 1 year of follow-up.

3. Study Design

3.1 Study Design and Rationale

This multicenter, open-label, phase 3b study will investigate the long-term safety and efficacy of rIX-FP for the routine prophylaxis and on-demand treatment of bleeding episodes in subjects with hemophilia B. Subjects will be eligible to enter the study if they have completed the phase I/III or phase III studies, or any other CSL-sponsored rIX-FP study and meet all other eligibility criteria. In addition, subjects who have not previously completed a CSL-sponsored rIX-FP lead-in study may be eligible to enter the study if they require major non-emergency surgery and meet all other eligibility criteria.

3.1.1 Routine Prophylaxis: First 6 Months

Arm 1: Subjects from all Lead-in Studies

During the first 6 months, subjects in Arm 1 will administer rIX-PP as routine prophylaxis either using the same treatment interval as in the lead-in study or using a different treatment interval of 7, 10, or 1.4 days, as determined by the investigator. The dose (35 to 75 IU/kg) of rIX-FP administered will be based on the subject's previous response to rIX-FP therapy and/or FIX trough activity (see Section 4.1.1) and dose guidelines in Section 3.2.1.3.

Arm 2

Subjects from a Lead-in Phase II/III Study Arm 2 Who have ≧26 Weeks Experience with rIX-FP Prophylaxis Therapy with a 7-Day Treatment Interval

During the first 6 months, subjects in this group will administer rIX-FP (75 IU/kg) as routine prophylaxis using a treatment interval of 14 days.

Arm 3

Upon completion of the surgery substudy, subjects in Arm 3 may begin the prophylaxis treatment period in the main study. During the first 6 months, subjects will administer rIX-FP as prophylaxis using a treatment interval of 7 days. The dose (35 to 50 IU/kg) of rIX-FP administered will be based on the subject's FIX trough activity (see Section 4.1.1).

3.1.2 Routine Prophylaxis: 6 to 36 Months

After completion of the initial 6-month treatment period, all subjects (i.e., Arms 1, 2 and 3) will administer rIX-FP as routine prophylaxis using a treatment interval of 7, 10, or 14 days for the remainder of the study. Subjects from lead-in phase II/III study Arm 2, who were using a 7-day treatment interval during the previous 6 months, will switch to a 14-day treatment interval for at least 6 months.

From 6 to 36 months of the study, subjects may use the same treatment interval or they may change treatment interval in consultation with the investigator at any of the subsequent 6-month follow-up visits (see Section 3.2.1.2). The dose of rIX-FP administered will be between 35 and 75 IU/kg (see Section 4.1.1).

Subjects ≧18 years of age may also administer rIX-FP as routine prophylaxis using a 21-day treatment interval at a dose of 100 IU/kg. Subjects <18 years of age are not permitted to use a treatment interval of 21 days.

Subjects who transfer to a 21-day treatment interval for the first time must have completed at least 6 months of prophylaxis treatment with a 14-day treatment interval and have undergone PK evaluation with 100 IU/kg rIX-FP (see Section 3.1.3).

3.1.3 Pharmacokinetic Evaluation of rIX-FP (100 IU/kg)

Subjects must undergo a PK evaluation with a single injection of rIX-FP (100 IU/kg) if either 1) they are in Arm 3 or 2) they intend to begin administering rIX-FP as routine prophylaxis using a 21-day treatment interval for the first time. The PK evaluation should be performed after a washout period of either at least 4 days for a current marketed FIX product (Arm 3) or at least 14 days for rIX-PP. Samples for PK evaluation will be collected before administration of rIX-FP, 30 minutes after the completion of the injection (to evaluate peak FIX activity level and incremental recovery) and at specified timepoints after injection.

The investigator may also choose to complete a PK assessment of 50, 75 or 100 IU/kg (as appropriate) rIX-FP with selected timepoints before starting surgical prophylaxis with rIX-FP (for subjects from lead-in studies), at the investigator's discretion or CSL's request, or in the event of (but not limited to) poor efficacy or suspicion of inhibitor development.

3.2 Dose and Dosing Regimen

3.2.1 Routine Prophylaxis Treatment

3.2.1.1 rIX-FP Treatment interval

During the first 6 months of the study, subjects or their caregivers will administer rIX-FP as routine prophylaxis using the following treatment intervals:
Arm 1: 7, 10, or 14 days.
Arm 2: 7 or 14 days,
Arm 3: 7 days.

For subjects in Arm 1, the treatment interval will be chosen by the investigator at the beginning of the study based on the subject's previous experience (i.e., during the lead-in study) and subject preference.

For subjects in Arm 2, the treatment interval will be based on the duration of their prophylaxis treatment in the lead-in study; a 14-day treatment interval will be assigned to subjects who completed at least 26 weeks of prophylaxis treatment during the lead-in study and a 7-day treatment interval will be assigned to subjects who did not complete at least 26 weeks of prophylaxis treatment during the lead-in study. Subjects in Arm 2 who start on a 7-day treatment interval and complete a total (i.e., during the lead-in study and this study) of at least 26 weeks of prophylaxis treatment, should then switch to a 14-day treatment interval for a period of at least 6 months.

Upon completion of the surgery substudy, subjects in Arm 3 may begin the prophylaxis treatment period in the main study. During the first 6 months, subjects in this group will administer rIX-FP as routine prophylaxis using a treatment interval of 7 days. The dose (35 to 50 IU/kg) of rIX-FP administered will be based on the subject's FIX trough activity (see Section 4.1.1). At the end of the initial 6 month period, subjects may remain using their current treatment or they may be switched to a 7, 10, or 14-day treatment interval. Subjects ≧18 years of age may also be switched to a 21-day treatment interval after completing at least 6 months of a 14-day prophylaxis regimen and a 100 IU/kg rIX-FP PK evaluation period (see Section 3.1.3).

3.2.1.2 Changing the rIX-FP Treatment Interval

During each 6 months of the treatment period, the treatment interval should not be changed unless deemed necessary by the investigator for the subject's safety. At the end of each 6-month period (i.e., at the 6 month, 12 month, 18 month, 24 month, and 30 month follow-up visits) the investigator may choose to change the treatment interval based on the their assessment of efficacy/safety, subject treatment compliance, and/or subject preference.

3.2.1.3 rIX-FP Dose

The dose of rIX-FP administered for routine prophylaxis will be based on the subject's previous experience (i.e., during a lead-in study) and/or the targeted FIX activity trough level (target FIX activity level >2%, but optimally, between 5 and 15%).

The maximum dose of rIX-FP for routine prophylaxis will be 50 IU/k per injection for subjects using a 7-day treatment interval and 75 IU/kg per injection for subjects using a 10 or 14-day treatment interval (Table 6), unless a higher dose for a given subject is approved by CSL. The dose of rIX-FP will be 100 IU/kg per injection for subjects using a 21-day treatment interval (Table 6). For all treatment intervals, the total dose of rIX-FP administered for routine prophylaxis over a 28-day period is not to exceed 250 IU/kg, without approval from CSL.

TABLE 6
Dose Guidelines for Prophylaxis Treatment

| | Treatment Interval | | | |
|---|---|---|---|---|
| | 7 days | 10 days¹ | 14 days | 21 days |
| Suggested | 25 to 50 | 50 to 75 | 75 | 100 |
| dose (IU/kg) | | | | |
| Maximum | 50² | 75 | 75 | 100 |
| dose (IU/kg) | | | | |

¹The 10-day treatment interval may be based on a schedule of once every 10 calendar days or 3 times a month (i.e., 1^{st}, 11^{th} and 21^{st} day of each month).
²An rIX-FP dose higher than 50 IU/kg is acceptable if the FIX activity trough level is <5% at Day 7 and a higher trough level is necessary to prevent spontaneous bleeding.

4. Dosing and Administration

4.1 Dosing and Administration

The investigator (or delegate) will administer or dispense rIX-FP only to subjects included in this study or their caregivers following the procedures set out in this study protocol. Subjects or their caregivers or qualified study personnel will administer the rIX-FP as a bolus IV injection.

When making the dose calculation, the total actual units (i.e., as per the prescribed dose) may be rounded up or down to target full vials (as actual 10), if possible, but the final dose needs to be within 10% of the prescribed dose. The dose of rIX-FP is based on the subject's most current body weight as recorded in the eCRF.

4.1.1 Adjustment of Dose for Prophylaxis Treatment

The dose of rIX-FP required for prophylaxis will be based on the subject's previous experience and/or the target FIX activity. The following formula will be used to calculate dose based on FIX activity required at peak:

$$\begin{matrix}Number \\ of factor \\ IX IU \\ required \\ ( IU )\end{matrix} = \begin{matrix}Body \\ weight \\ ( kg )\end{matrix} \times \begin{matrix}Desired \\ factor IX \\ increase \\ ( \% or \\ IU \text{/} dL )\end{matrix} \times \begin{matrix}Reciprocal of \\ observed \\ recovery \\ ( IU \text{/} kg per \\ IU \text{/} dL )\end{matrix}$$

The target trough FIX activity for routine prophylaxis is greater than 2%, but optimally, between 5 and 15% above baseline.

5. Pharmacokinetic and Pharmacodynamic

5.1 Pharmokinetic Analysis

5.1.1. Trough Factor IX Activity Level

During the study, blood samples will be taken for the assessment of trough FIX activity level at each major bleeding episode (if feasible) and the specified follow-up visits.

5.1.2 Pharmacokinetic Evaluation of rIX-FP

In addition, subjects beginning routine prophylaxis treatment using a treatment interval of

21 days for the first time and subjects in Arm 3, will undergo a PK evaluation with a single injection of rIX-FP (100 IU/kg). For this PK evaluation, blood samples will be taken for the measurement of the FIX activity level at the following time points (required or optional) after injection:
30±5 minutes
72±24 hours (i.e., 3±1 days)
168±24 hours (i.e., 7±1 days)
336±24 hours (i.e., 14±1 days)
504±24 hours (i.e., 21±1 days)

If during the PK assessment period, a subject experiences a bleeding episode, no further blood samples will be taken as part of PK assessment, regardless of whether the bleeding episode is treated or not. The PK may or may not need to be repeated.

A PK evaluation of 50, 75, or 100 IU/kg rIX-FP (with selected time point[s]) may also be assessed at the investigator's discretion or CSL's request, in the event of (but not limited to) poor efficacy, suspicion of inhibitor development, or before major surgery.

The incremental recovery and FIX activities will be reported. Additional PK parameters (e.g., AUC, t_{1/2}) may be calculated, if deemed appropriate.

### Example 3

### A Phase 3b Study of a Recombinant Coagulation Factor IX Albumin Fusion Protein (rIX-FP) in Subjects with Hemophilia B with a 21-Day Dosing Interval

Subjects ≧18 years of age may administer rIX-FP as routine prophylaxis using a 21-day treatment interval at a dose to 100 IU/kg. Initially, they must undergo a PK evaluation period with a single injection of rIX-FP (100 IU/kg) after a rIX-FP washout period of at least 14 days.

In order to participate in the 21-day treatment regimen, the following criteria must be met:
1. Subjects at least 18 years of age.
2. Completed at least 6 months of routine prophylaxis with rIX-FP on a 14-day treatment interval regimen in a phase II/III or phase III study.
3. Complete a 21-day pharmacokinetic evaluation of 100 IU/kg rIX-FP.

Additionally, the investigator should assess the following prior to changing the interval treatment:
1. The efficacy and safety
2. Subject treatment compliance
3. Subject preference

Therefore, a subject who is eligible for the 21-day treatment regimen will have been on routine prophylaxis for at least a year (7-day treatment Interval for at least 6 months in a lead-in study, and 14-day treatment interval in a lead-in study or the 3001 study). A PK of 100 IU/kg rIX-FP will be completed before a subject will be able to switch to a longer treatment interval, allowing evaluation of the subject's trough level at 21 days prior to starting prophylaxis at that dose.

Prophylaxis was conceived from the observation that moderate hemophilia patients with clotting factor level >1 IU/dl (>1%) seldom experience spontaneous bleeding and have much better preservation of joint function. Therefore, prophylaxis regimen to maintain FIX activity level above 1% is reviewed as a surrogate efficacy assessment of FIX replacement therapy. A PK modeling based on the PK data from the two completed rIX-FP clinical studies indicate that 68% of study subjects may have 2% FIX activity above baseline, 95% of subjects may have 1% FIX activity above baseline at Day 21 post administration of 100 IU/kg rIX-FP, see also FIG. 7.

TABLE 7
Predicted PK values for Different rIX-FP Dosing Regimens

| | 40 IU/kg QW | 75 IU/kg Q2W | 100 IU/kg Q3W |
|---|---|---|---|
| FIX Actvity | | | |
| Cmax (%) | 49-89 | 76-149 | 97-191 |
| Cmin (%) | 7.2-21 | 2.5-11 | 0.9-5.8 |
| Patients with: | | | |
| Cmin >2% | >95 | >95 | 68 |
| Cmin >5% | >95 | 58 | 11 |

Cmax/min = maximum or minimum FIX activity level

In agreement with the PK modeling, in one of the completed Phase II/III study, two study subjects had participated 50 IU/kg of rIX-FP PK assessment beyond Day 14: one subject had FIX activity level of 2.1% above baseline (FIX activity prior to rIX-FP) at Day 19, another subject had FIX activity level of 1.7% above baseline at Day 21 post 50 IU/kg rIX-FP injection.

It is desirable that the majority of patients with hemophilia will have the trough level above 1% at Day 21-post 100 IU/kg rIX-PP injection and may be candidate for the 21 day prophylaxis regimen. Nevertheless, optimal treatment regimen must incorporate clinical outcome (such as the clinical response, subject's physical activity, bleeding frequency, joint status, quality of life or desired to have higher trough) which may be the important decision factors when choose the treatment interval.

A study with a 100 IU/kg rIX-FP dosing regimen was carried out with about with five patients having hemophilia. The FIX activity (IU/dL in %) was measured after 0 h, 0.5 h, 72 h (3 days), 168 h (7 days), 336 h (14 days), and 504 h (21 days). The prophylaxis regimen shows a FIX activity of at least 4.1% at day 21-post 100 IU/kg rIX-PP injection. The results are summarized in the Table 8 below.

TABLE 8
FIX activity (IU/dL in %) for 100 IU/kg rIX-FP Dosing Regimen.

| | | | 72 h | 168 h | 336 h | 504 h |
|---|---|---|---|---|---|---|
| | 0 h | 0.5 h | (3 days) | (7 days) | (14 days) | (21 days) |
| Patient 1 | 10.8 | 102.9 | 58.1 | 33.9 | 14.3 | 5.2 |
| Patient 2 | 1.2 | 105.5 | 47.9 | 33.3 | 14.2 | 4.9 |
| Patient 3 | 2.7 | 117 | 50 | 27.8 | 7.4 | 4.1 |
| Patient 4 | 3.6 | 120.2 | 52.9 | 26.5 | | |
| Patient 5 | 0 | 108.8 | 47.7 | | | |
| Mean | 5.82 | 110.88 | 51.32 | 30.35 | 11.96667 | 4.733333 |
| SD | 4.72288 | 6.652338 | 3.87319 | 3.237669 | 3.229379 | 0.46428 |
| N | 5 | 5 | 5 | 4 | 3 | 3 |

Mean = mean value based on measured values and the number of participants, SD = standard deviation, N = number of patients.

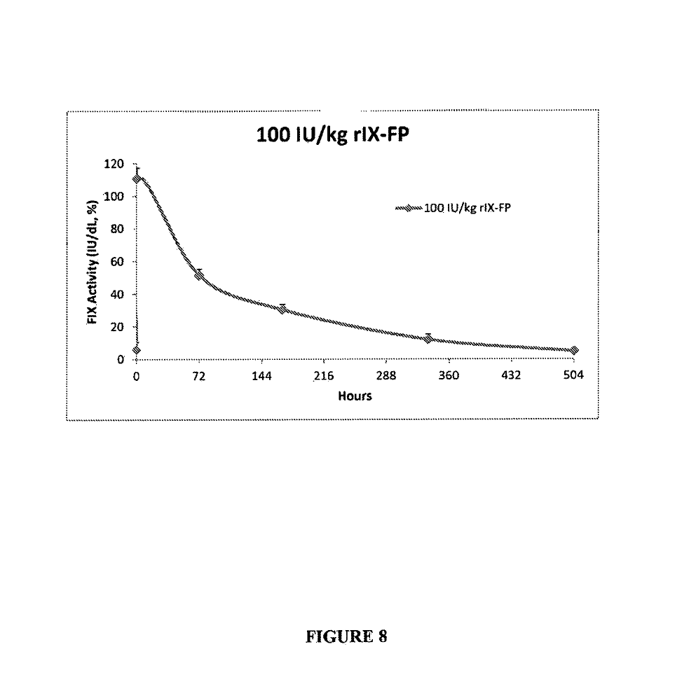

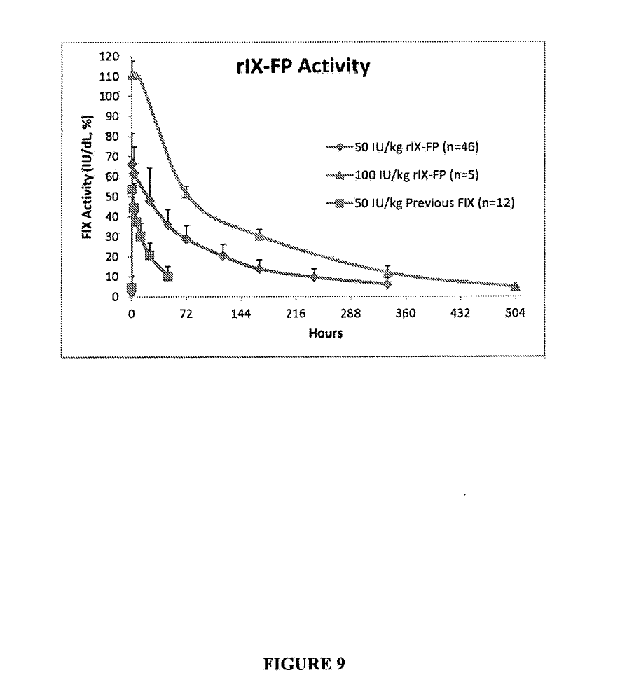

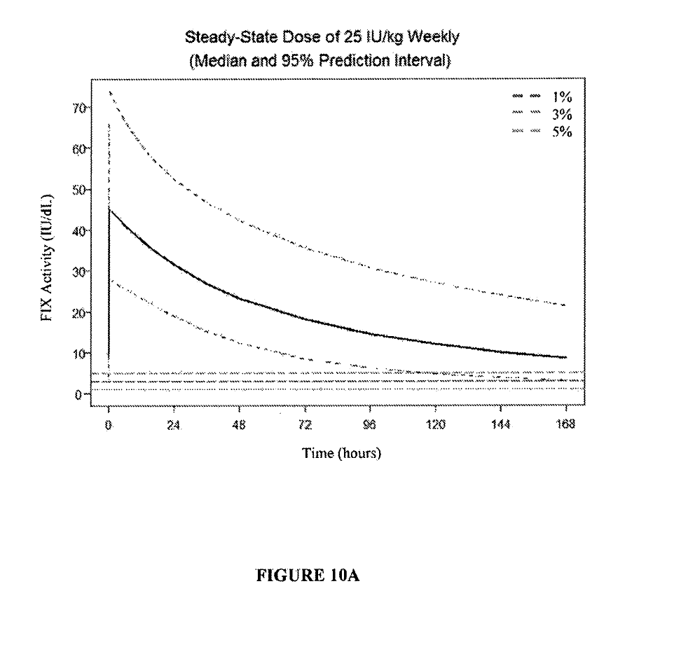

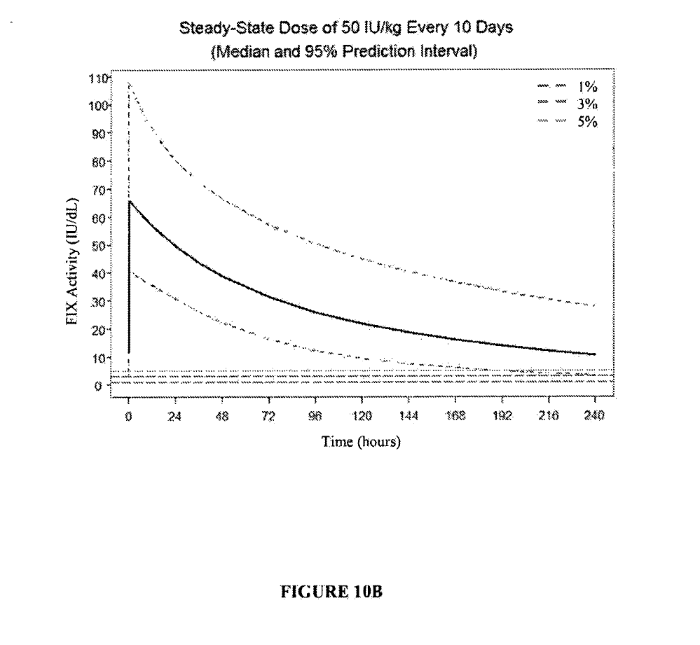

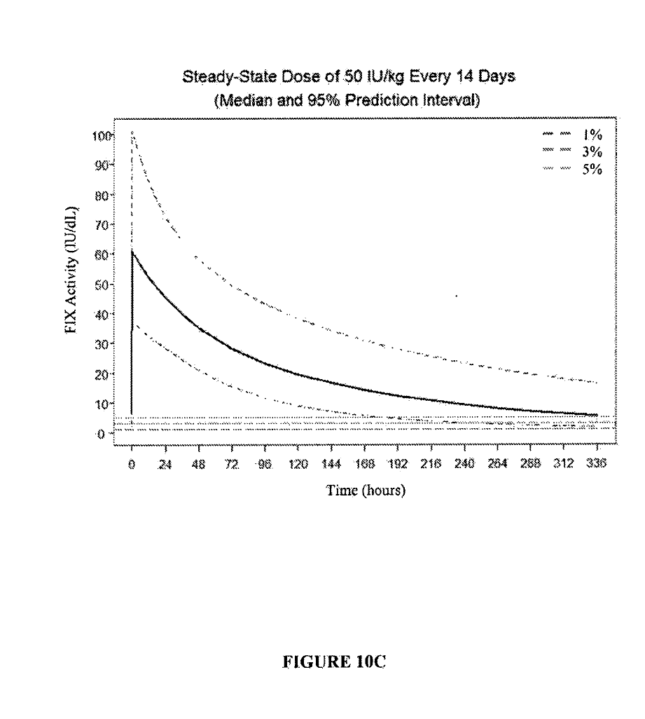

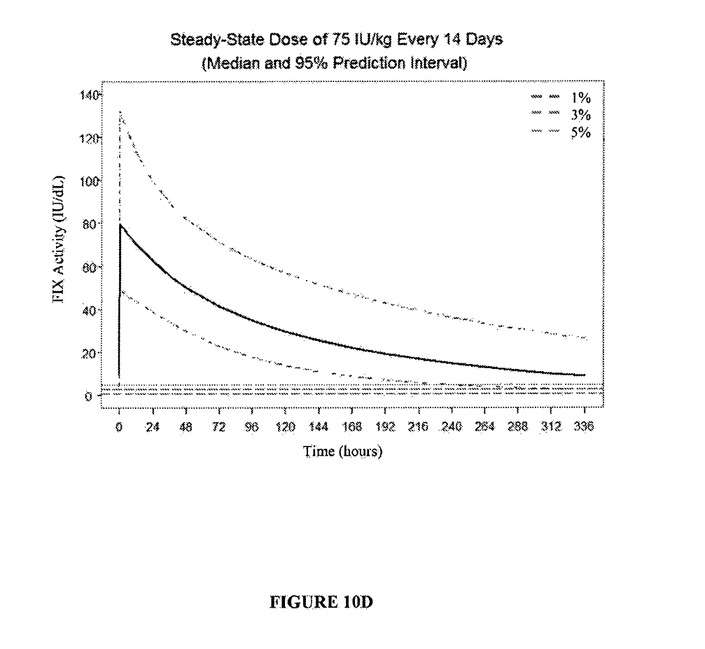

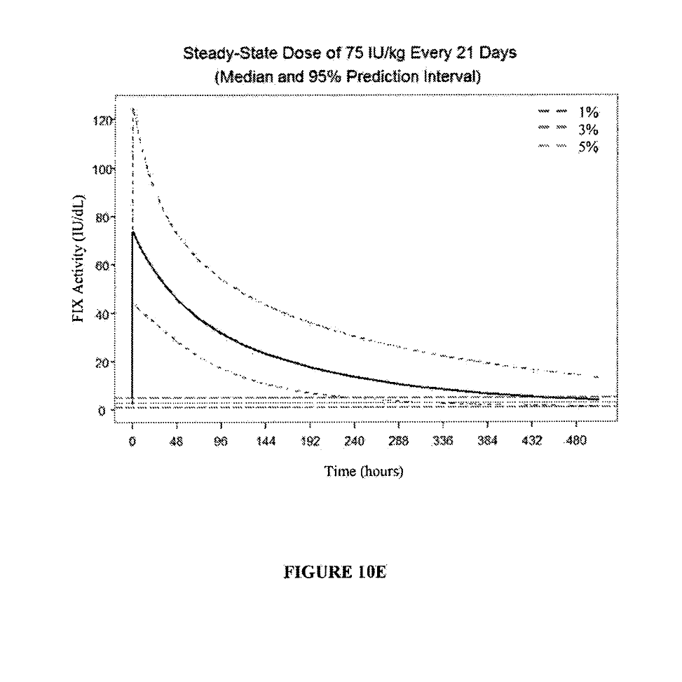

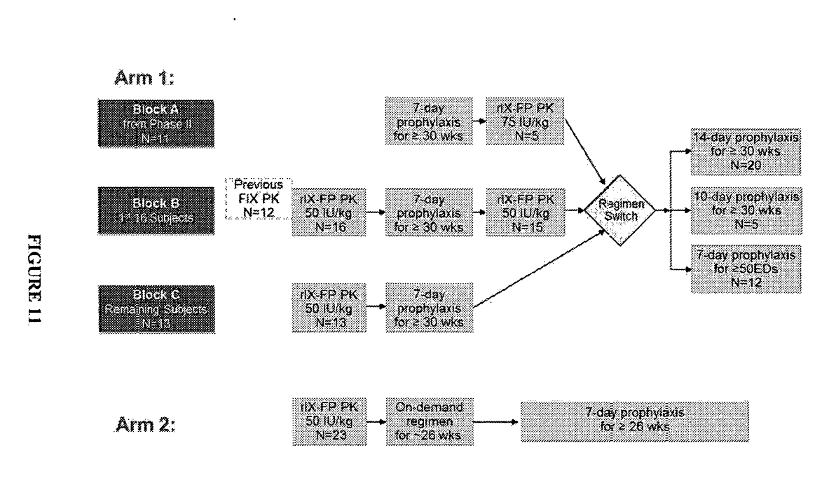

A plot of the FIX activity values (IU/dL in %) over time is shown in FIG. 8. A comparison of these values with corresponding values when applying 50 IU/kg rIX-FP dosing regimen and when applying 50 IU/kg rFIX (BeneFIX®) is shown in FIG. 9.

This Phase 3b study aims to evaluate the long-term safety and efficacy of rIX-FP for routine prophylaxis in subjects who participated in two Phase III registration studies, or any other CSL-sponsored rIX-FP lead-in study. At the end of the study, subjects ace expected to have accumulated at least 100 FIX-FP exposure days (EDs) during enrollment in all CSL-sponsored rIX-FP studios. We intend to allow more flexible prophylaxis regimens that include the regimens patients and their physicians may choose.

### Example 4

### Population PK Modeling

Based on PK data from previous clinical trials, a population pharmacokinetic analysis of recombinant factor IX albumin fusion protein in subjects with Hemophilia B was conducted in order to characterize the population PK in subjects with hemophilia B, to identify variability and potential determinants (demographic and clinical covariates) of PK variability, and to simulate single and steady-state FIX activity-time profiles for various dosing scenarios.

Simulations were carried out in order to determine for how long FIX activity maintains at a trough level above 1%, 3% and 5% after a single dose of 25 IU/kg, 50 IU/kg, or 75 IU/kg. The results are summarized in Table 9a.

TABLE 9a
Simulated Durations that Exogenous FIX Activity
is maintained above 1, 3 and 5%.

| Simulated | Duration | Duration | Duration |
|---|---|---|---|
| Single | above | above | above |
| IV Dose | 1% | 3% | 5% |
| 25 IU/kg | 16 days | 10 days | 7 days |
| 50 IU/kg | 21 days | 15.5 days | 12 days |
| 75 IU/kg | 27 days | 19.5 days | 16 days |

Simulated steady-state trough exogenous FIX activity levels are summarized for a number of dosing regimens in Table 96.

TABLE 9b
Summary of the Simulated Trough Exogenous FIX Activity.

| | Median (95% Prediction Interval) |
|---|---|
| Simulation | (IU/dL) |
| 25 IU/kg weekly | 7.4 (2.0-19.8) |
| 50 IU/kg every 10 days | 9.3 (2.2-26.3) |
| 50 IU/kg every 14 days | 4.5 (0.7-15.2) |
| 75 IU/kg every 14 days | 7.9 (1.5-25.2) |
| 75 IU/kg every 21 days | 2.7 (0.3-12.0) |

The term median refers to the middle value, calculated on all data. The values in the parenthesis are the 5% and 95% bounds.

TABLE 9c
Dosage Guidelines.

| | Treatment Interval | | | |
|---|---|---|---|---|
| | 7 days | 10 days¹ | 14 days | 21 days |
| Suggested | 25 to 40 | 50 to 75 | 50 to 75 | 100 |
| dose (IU/kg) | | | | |
| Maximum | 40² | 75 | 75 | 100 |
| dose (IU/kg) | | | | |

¹The 10-day treatment interval may be based on a schedule of once every 10 calendar days or 3 times a month (i.e., 1^{st}, 11^{th} and 21^{st} day of each month).
²An rIX-FP dose higher than 40 IU/kg is acceptable if the FIX activity trough level is <5% at Day 7 and a higher trough level is necessary to present spontaneous bleeding.

### Example 5

### FIX Activity Level Observed in it Phase II/III Clinical Study of a Recombinant Coagulation Factor IX Albumin Fusion Protein (rIX-FP) in Subjects with Hemophilia B

This Phase II/III study was an open-label study to evaluate the efficacy-pharmacokinetics and safety of rIX-FP, which was developed for the prophylaxis (arm 1 of the study) and treatment of bleeding episodes (arm 2 of the study) in patients with congenital Factor IX (FIX) deficiency (hemophilia B). The study consisted of a screening period of less than one month, a 14 day pharmacokinetic (PK) evaluation period, followed by approximately a 14-month safety and efficacy evaluation period with rIX-FP.

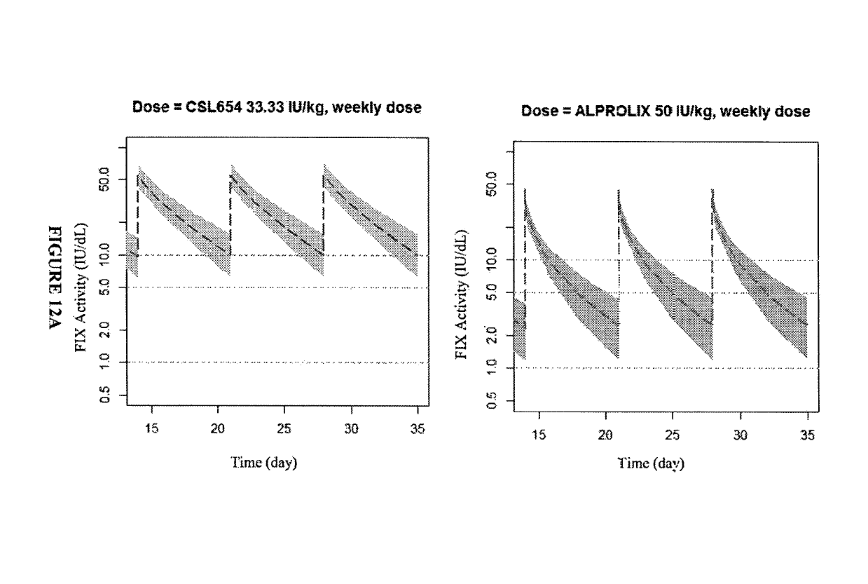

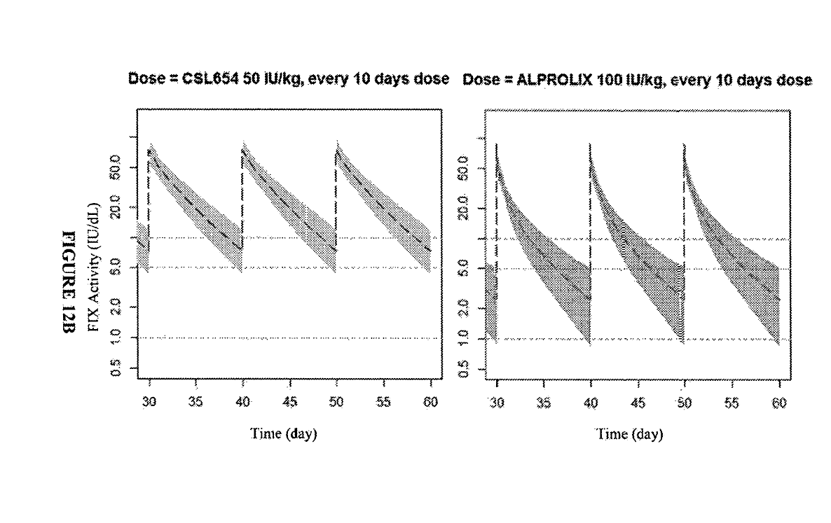

The full PK of rIX-FP was evaluated at the beginning of the study. In addition, or the beginning of the study, the pharmacokinetics (PK) of the previous. FIX product (plasma-derived or recombinant) was evaluated in a subset of subjects. A flow chart of the study including both study arms, the prophylaxis and the on-demand arms, is shown in FIG. 11.

Prophylaxis Subjects (Arm 1):

Approximately 40 subjects began a 14-month treatment period of prophylaxis, which consisted of two halves, and subjects received one or two treatment regimens of rIX-FP. Subjects were initially treated with a prophylactic dose of rIX-FP of 35-50 IU/kg, and the dose was adjusted up to a maximum of 75 IU/kg, as specified in the protocol, with a target of maintaining the trough FIX activity level above 1% between doses. The treatment interval of 7 days was maintained during the first segment (26-30 weeks) of the study. During the second half of the study, a fraction of the subjects switched to a prophylactic therapy regimen with a treatment interval of 10 or 14 days for at least 30 weeks at a dose of 75 IU/kg of rIX-FP.

The results of the phase II/phase III study indicated an excellent safety profile for rIX-FP. No subject developed FIX inhibitor, rIX-FP antibodies or CHO host cell protein antibodies. Adverse reactions were reported in only 7.9% of the subjects, and anaphylaxis was not observed. In conclusion, rIX-FP is effective for control and prevention of bleeding episodes, routine prophylaxis and perioperative prophylaxis.

By using the rIX-FP of the current Invention, the FIX consumption per month is reduced when compared the monthly doses of previous FIX (BeneFIX®), see Table 10. When applying the 10 or the 14-day dosing interval of 50 IU/kg rIX-FP, the monthly consumption was even further reduced, which indicates a high stability of the rIX-FP of this invention.

TABLE 10
Monthly consumption of rIX-FP with a treatment interval of 7, 10 or 14 days
with the doses of rIX-FP, as displayed in Tables 8, 9, and 10. Comparison
with the monthly consumption level of previous FIX (BeneFIX ®).

| | 7 d | 10 d | 14 d | Previous FIX |
|---|---|---|---|---|
| Monthly IU/kg | N = 40 | N = 7 | N = 21 | N = 28 |
| Mean | 202 | 201 | 157 | 320 |
| Median | 195 | 222 | 162 | |
| IQR | 167, 215 | 149, 224 | 159, 164 | 208, 365 |

IQR: interquartile range. Mean refers to the average, value calculated by addition of the monthly rIX-FP consumption values and division by the number of patients, whereas median means that half of the study objects have a higher, and half of the study objects have a lower monthly rIX-FP consumption.

On-Demand Subjects (Arm 2):

Approximately 25 on-demand subjects were enrolled in the study, which consisted of a one month screening period, approximately 26 weeks of on-demand treatment (only receives rIX-FP after bleeding episode), followed by prophylactic weekly therapy with rIX-FP. The annualized bleeding rate of on-demand treatment was compared to the prophylactic treatment.

During this study, subjects visited study centers for assessment, including FIX activity level test, every 4±1 weeks. Therefore, we are able to collect FIX activity level throughout the study.

Majority of the on-demand subjects required treatment (due to bleed) more than once a month. However, two of the on-demand subjects had longer than one month between the treatments that made it possible to test the FIX activity at 28 days from the previous rIX-FP treatment (Table 11).

TABLE 11
FIX activity at baseline and at Day 28

| | FIX activity | FIX activity at Day 28 |
|---|---|---|
| | (% or IU/dL) | post 50 IU/kg |
| | at baseline | rIX-FP injection |
| Subject #1 | 0.3 | 3.4 |
| Subject #2 | 0.4 | 0.8 |

Prophylaxis was conceived from the observation that moderate hemophilia patients with clotting factor level >1 IU/dl (>1%) seldom experience spontaneous bleeding and have much better preservation of joint function. Therefore prophylaxis regimen to maintain FIX activity level (plasma level) above 1% it targeted for the FIX replacement therapy. This preliminary data indicated that a dose higher than 50 or 100 IU/kg of rIX-FP may be sufficient for proving 1% FIX activity above baseline at day 28 post rIX-FP injection in subjects with hemophilia, that implicated a monthly dosing interval may be feasible.

A PK modeling based on the PK data from the completed rIX-PP phase II/III clinical study was conducted and compared with corresponding PK modelling data based on available ALPROLIX™ kinetic data.

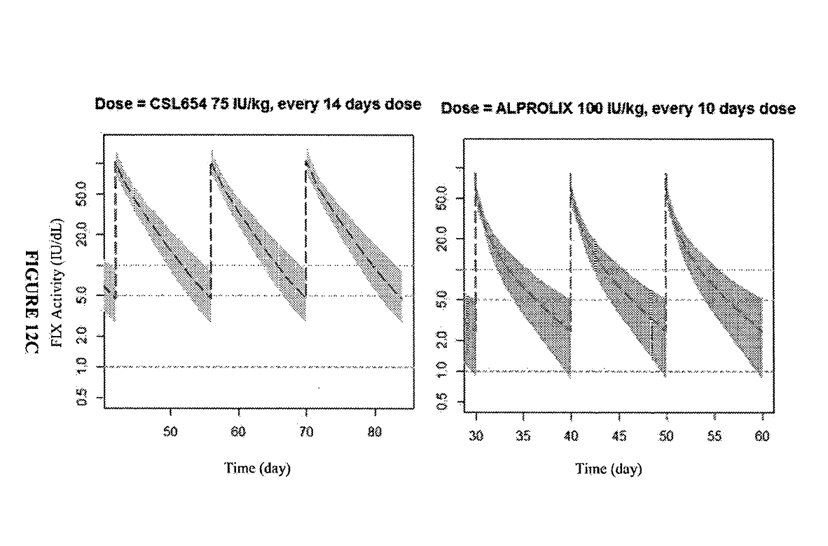

A PK modeling based on the PK data from the completed rIX-FP phase II/III clinical study indicated that with a weekly dosing interval with 33.33 IU/kg rIX-FP, 50% of study subjects bad a trough of 10% FIX activity above baseline, see FIG. 12 A (left). An analogous PK modeling was conducted, based on the ALPROLIX™ PK kinetic data. When a weekly dosing interval of 50 IU/kg ALPROLIX™ was applied, 50% of study subjects had a trough of 2% FIX activity above baseline, see FIG. 12 A (right.

Another PK modeling based on the PK data from the completed rIX-FP phase II/III clinical study indicated that with a dosing interval of 10 days with 50.0 IU/kg rIX-FP, 50% of study subjects had a trough of 7.5% FIX activity above baseline, see FIG. 12 B (left). An analogous PK modeling was conducted, based on the ALPROLIX™ PK kinetic data. When a dosing interval of every 10 days with 100 IU/kg ALPROLIX™ was applied, 50% of study subjects had a trough of 2% FIX activity above baseline, see FIG. 12 B (right).

Yet another PK modeling based on the PK data from the completed rIX-FP phase II/III clinical studies indicated that with a dosing interval of 14 days with 75.0 IU/kg, 50% of study subjects had a trough of 5% FIX activity above baseline, see FIG. 12 C (left). An analogous PK modeling was conducted, based on the ALPROLIX™ PK kinetic data. When a dosing interval of every 10 days with 100 IU/kg ALPROLIX™ was applied, 50% of study subjects had a trough of 2% FIX activity above baseline, see FIG. 12 C (right).

### Example 6

### PK Parameters Derived from Concentration Decay Data of rIX-FP or rIX-Fc (Alprolix™) Obtained from Human Plasma after Administering 50 IU/Kg of rIX-FP or rIX-Fc (Alprolix™) to Human Subjects

Approximately 28 subjects were injected with a 50 IU/kg dose of rIX-FP. The activity of rIX-FP in human plasma was measured over time. The decay kinetics of rIX-FP was analyzed, from which the pharmacokinetic parameters shown in Table 12 were determined. In particular, Table 12 displays the arithmetic mean value and the corresponding coefficient of variation (CV) of the pharmacokinetic parameters.

The pharmacokinetic parameters of rIX-Fc (Alprolix™) shown in Table 12 are derived from a study with 22 patients as disclosed in the Alprolix™ FDA prescribing information.

TABLE 12
Comparison of pharmacokinetic parameters of rIX-FP and rIX-Fc
(Alprolix ™)

| | rIX-FP | ALPROLIX ™ |
|---|---|---|
| | (50 IU/kg) | (50 IU/kg) |
| | with baseline | FDA prescribing |
| Mean pK | correction; | information |
| parameters | Prophylaxis | rFIXFc |
| (CV values in %) | N = 28 | (N = 22) |
| AUC_{0-inf}(IU*h/dL) | 6203.67 | 1619.1 |
| | (29.8) | (26.1) |
| | | 1. |
| t½ (h) | 87.0538 | 86.52 |
| | (28.8) | (37.2) |
| | | 2. |
| CL/BW (mL/h/kg) | 0.8839 | 3.304 |
| | (33.6) | (28.4) |
| | | 3. |
| Incremental | 1.2859 | 1.0154 |
| Recovery | (26.0) | (58.7) |
| ((IU/dL)/(IU/kg)) | | 4. |
| Vss/BW (dL/kg) | 1.0097 | 3.270 |
| | (21.6) | (28.2) |
| | | 5. |
| MRT (h) | 121.3904 | 101.96 |
| | (26.7) | (29.7) |

Abbreviations: AUCinf = area under the FIX activity time curve; t½ = elimination half-life; CL/BW = bodyweight adjusted clearance; Incremental recovery = defined as IU/dl rise in plasma per IU/kg administered, Vss/BW = bodyweight adjusted volume of distribution at steady-state, MRT = mean: residence time, N = approximate number of human subjects.

According to the above data, rIX-FP has an about 3.8-fold higher AUC0-inf, an about 3.7-fold reduced CL/BW, an about 26% higher incremental recovery, an about 3.2-fold lower volume of distribution, and an increased mean residence time of the drug by about 19%, when compared to rIX-Fc (ALPROLIX™). These PK values indicate an improved activity and stability of Factor IX fusion protein of this invention (rIX-PP).

1.-115. (canceled)

116. A method of preventing bleeding in a subject, comprising prophylactically administering to the subject a fusion protein comprising
a) Factor IX (FIX) and
b) human albumin,
wherein the FIX is connected to the N-terminus of human albumin via a peptide linker cleavable by proteases involved in coagulation or activated by coagulation enzymes, and wherein the fusion protein is administered intravenously at a dose of about 25-50 IU/kg and a dosing interval of once about every 6 to 8 days.

117. The method according to claim 116, wherein the dose is about 35-50 IU/kg.

118. The method according to claim 116, wherein the dose is about 25-40 IU/kg.

119. The method according to claim 116, wherein the dose is about 40, 45 or 50 IU/kg.

120. The method according to claim 116, wherein the dosing interval is once about every 7 days.

121. The method according to claim 116, wherein a FIX plasma level trough of at least about 1%, at least about 2%, or between 5 and 15%, above baseline is maintained for the entire dosing interval.

122. The method according to claim 116, wherein the FIX is a human FIX.

123. The method according to claim 116, wherein the peptide linker is cleavable by FIXa and/or by FVIIa/Tissue Factor (TF).

124. The method according to claim 123, wherein the peptide linker comprises an amino acid sequence selected from SEQ ID NO: 2 and SEQ ID NO: 3.

125. The method according to claim 116, wherein the fusion protein has an amino acid sequence at least 70% identical to the amino acid sequence set forth in SEQ ID NO: 1.

126. The method according to claim 116, wherein the fusion protein comprises the amino acid sequence set forth in SEQ ID NO: 1.

127. The method according to claim 116, wherein the subject is a human.

128. The method according to claim 127, wherein the human suffers from hemophilia B.

129. (canceled)

130. The method according to claim 116, wherein the fusion protein is administered at a concentration of about 100 to 400 IU/ml.

131. A method of preventing bleeding in a subject, comprising prophylactically administering to the subject a fusion protein comprising
a) Factor IX (FIX) and
b) human albumin,
wherein the FIX is connected to the N-terminus of human albumin via a peptide linker cleavable by proteases involved in coagulation or activated by coagulation enzymes, and wherein the fusion protein is administered intravenously at a dose of about 50-75 IU/kg and a dosing interval of once about every 10 to 14 days.

132. The method according to claim 131, wherein the dosing interval is once about every 14 days.

133. The method according to claim 131, wherein the dose is about 75 IU/kg and the dosing interval is once about every 14 days.

134. The method according to claim 131, wherein the FIX is a human FIX.

135. The method according to claim 131, wherein the peptide linker is cleavable by FIXa and/or by FVIIa/Tissue Factor (TF).

136. The method according to claim 135, wherein the peptide linker comprises an amino acid sequence selected from SEQ ID NO: 2 and SEQ ID NO: 3.

137. The method according to claim 131, wherein the fusion protein has an amino acid sequence at least 70% identical to the amino acid sequence set forth in SEQ ID NO: 1.

138. The method according to claim 131, wherein the fusion protein comprises the amino acid sequence set forth in SEQ ID NO: 1.

139. The method according to claim 131, wherein the subject is a human.

140. The method according to claim 139, wherein the human suffers from hemophilia B.

141. (canceled)

142. The method according to claim 131, wherein the fusion protein is administered at a concentration of about 100 to 400 IU/ml.

143. A method of preventing bleeding in a subject, comprising prophylactically administering to the subject a fusion protein comprising
a) Factor IX (FIX) and
b) human albumin,
wherein the FIX is connected to the N-terminus of human albumin via a peptide linker cleavable by proteases involved in coagulation or activated by coagulation enzymes, and wherein the fusion protein is administered intravenously at a dose of about 90-250 IU/kg and a dosing interval of once about every 3 weeks or longer.

144. The method according to claim 143, wherein the dose is about 100 IU/kg.

145. The method according to claim 143, wherein a FIX plasma level trough of at least about 2-4% above baseline is maintained for the entire dosing interval.

146. The method according to claim 143, wherein the dosing interval is once about every 21 days.

147. The method according to claim 143, wherein the FIX is a human FIX.

148. The method according to claim 143, wherein the peptide linker is cleavable by FIXa and/or by FVIIa/Tissue Factor (TF).

149. The method according to claim 148, wherein the peptide linker comprises an amino acid sequence selected from SEQ ID NO: 2 and SEQ ID NO: 3.

150. The method according to claim 143, wherein the fusion protein has an amino acid sequence at least 70% identical to the amino acid sequence set forth in SEQ ID NO: 1.

151. The method according to claim 143, wherein the fusion protein comprises the amino acid sequence set forth in SEQ ID NO: 1.

152. The method according to claim 143, wherein the subject is a human.

153. The method according to claim 152, wherein the human suffers from hemophilia B.

154. (canceled)

155. The method according to claim 143, wherein the fusion protein is administered at a concentration of about 100 to 400 IU/ml.


SEQUENCE LISTING (4 sequences)

SEQ ID NO: 1 - protein, 1018 aa, Artificial Sequence
  rFIX-albumin fusion protein
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTPVSQTSKLTRAETVFPDVDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 2 - protein, 18 aa, Artificial Sequence
  linker
PVSQTSKLTRAETVFPDV

SEQ ID NO: 3 - protein, 19 aa, Artificial Sequence
  linker
PSVSQTSKLTRAETVFPDV

SEQ ID NO: 4 - protein, 7 aa, Artificial Sequence
  non-cleavable linker
GGGGGGV